(19) United States
(12) Patent Application Publication
(10) Pub. No.: US 2023/0101020 A1
(43) Pub. Date: Mar. 30, 2023

(54) Disposable PAPR with Viral Resistant Coatings

(71) Applicant: ABC Filtration Corp., Golden, CO (US)

(72) Inventors: Randall ALLARD, Golden, CO (US); Polly ALLARD, Golden, CO (US); Thomas J. McLEER, Laguna Niguel, CA (US); Philip J. TAMMINGA, Denver, CO (US)

(21) Appl. No.: 17/960,075

(22) Filed: Oct. 4, 2022

Related U.S. Application Data
(63) Continuation of application No. PCT/US2021/026044, filed on Apr. 6, 2021.
(60) Provisional application No. 63/062,374, filed on Aug. 6, 2020.
(60) Provisional application No. 63/052,101, filed on Jul. 15, 2020.
(60) Provisional application No. 63/006,457, filed on Apr. 7, 2020.

(51) Int. Cl. A62B 18/00; A62B 18/08; A61L 9/20

(52) U.S. Cl. CPC A62B 18/006; A62B 18/084; A61L 9/20; A61L 2209/14

(57) ABSTRACT

The PAPR kit is an air-purifying respirator that uses a blower to force the ambient air through air-filtration media to the hood assembly to create positive air pressure. The PAPR kit is module in design and can be employed in multiple use configurations and applications. The PAPR kit comprises a PAPR unit, the PAPR unit may be removably mounted or coupled to a back region of a hood assembly and/or a waist belt. The PAPR kit may further comprise an integrated or removable battery pack that is easily retained and/or removed from the PAPR unit.

### CROSS-REFERENCE TO RELATED APPLICATIONS

This application is a continuation of PCT Patent Application No. PCT/US2021/026044, entitled “IMPROVED PAPR WITH VIRAL RESISTANT COATINGS,” filed Apr. 6, 2021, which in turn claims the benefit of U.S. Provisional Appl. No. 63/006,457, filed Apr. 7, 2020, and entitled “Disposable PAPR with Viral Resistant Coatings;” U.S. Provisional Appl. No. 63/052,101, filed Jul. 20, 2020, entitled “Improved PAPR with Viral Resistant Coatings;” and U.S. Provisional Appl. No. 63/062,374, filed Aug. 6, 2020, entitled “Improved PAPR with Viral Resistant Coatings,” all of which are incorporated herein in their entireties.

### TECHNICAL FIELD

This invention relates to an improved PAPR kit that is compact, lightweight and disposable. More specifically, this invention relates to an improved PAPR kit comprising a germicidal or anti-viral coating that is compact, lightweight and disposable.

### BACKGROUND

Currently, there are variety of PAPR kits on the market that contain a variety of limitations and factors when considering their use in a variety of settings. Such limitations include (1) a plurality of parts that difficult to assemble and dissemble for use and decontamination; (2) there may be a reduced ability to hear due to the blower noise from the blower motor; (3) a significantly increased weight; they require a significant amount of storage space in between shifts; (4) difficult to maintain and properly disinfect the traditional PAPR kits—facilities must train staff to cleaning, disinfection & battery management procedures; and (5) potential exposure to live virus due to PAPR filters being reused (e.g., traditional PAPR filters are generally re-used until they are soiled, damaged or have reduced air flow).

### BRIEF DESCRIPTION OF THE DRAWINGS

FIGS. 1A-1B depict isometric views of one embodiment of a PAPR unit;

FIG. 2 depicts an isometric exploded view of a PAPR unit of FIGS. 1A-1B;

FIGS. 3A-3E depicts an isometric view of an alternate embodiment of a disposable PAPR unit;

FIG. 3F depicts an isometric exploded view of the PAPR unit of FIGS. 3A-3E;

FIGS. 4A-4B depicts isometric views an alternate embodiment of a PAPR unit;

FIGS. 5A-5E depicts various views of an alternate embodiment of a PAPR kit;

FIG. 6 depicts an isometric exploded view of the PAPR unit of FIGS. 5A-5E;

FIGS. 7A-7G depicts various views of one embodiment of a housing compartment;

FIGS. 8A-8G depicts various views of one embodiment of a blower motor;

FIGS. 9A-9H depicts various views of one embodiment of a filter cap base;

FIGS. 9I-9L depicts various views of an alternate embodiment of a filter cap base;

FIGS. 10A-10E depicts various views of one embodiment of a filter grate;

FIGS. 11A-11G depicts various views of one embodiment of a suspension frame;

FIGS. 12A-12C depicts various views of one embodiment of an adjustable collar;

FIGS. 13A-13D depicts isometric views of alternate embodiments of flexible hoods;

FIGS. 14A-14C depicts isometric views of alternate embodiments of rigid hoods;

FIGS. 15A-15C depicts various views of an alternate embodiment of a PAPR unit;

FIGS. 16A-16F depicts isometric views of an alternate embodiment of a PAPR kit;

FIGS. 17A-17D depicts various views of one embodiment of a PAPR kit of FIGS. 16A-16F;

FIGS. 18A-18B depict a front and side view of one embodiment of a filter assembly;

FIGS. 19A-19B depict isometric views of the filter assembly of FIGS. 18A-18B;

FIGS. 20A-20E depict various views of one embodiment of a filter cap plate;

FIGS. 21A-21F depict a front and side view of one embodiment of a filter cap base; and

FIGS. 22A-22B depict a front and isometric view of an alternate embodiment of a PAPR kit.

### BRIEF SUMMARY OF INVENTION

In one embodiment, the PAPR kit comprises a waist-fitted PAPR kit. The waist-fitted PAPR kit comprises a hood assembly, a PAPR unit, a belt, an at least one elongated air hose. The PAPR kit may further comprise a battery charger kit. The hood assembly may comprise a hood. The PAPR kit, the PAPR unit and/or hood assembly may further comprise at least one of a head suspension frame, at least one light and/or a collar. The hood may comprise a rigid or a flexible hood. The hood may comprise a tight-fitting hood or a loose-fitting hood. The flexible hoods may comprise a head cover, a head cover with shroud, head cover with double shroud, a headcover sealed seam hood. The rigid hood may comprise a helmet, a helmet with shroud and a helmet with a double shroud. The hood and/or the hood assembly may comprise a pathogen inactivation coating. The pathogen inactivation coating may comprise a germicidal, a solvent/detergent, a methylene blue, a amotosalen & UV, a riboflavin & UV coating, and/or any combination thereof. The PAPR unit comprises a blower motor, a battery pack and at least one filter media. At least a portion of the PAPR unit coupled to at least a portion of the belt. The PAPR unit may further comprise at least one or more of a housing, a low voltage alarm, an air flow sensor, a toggle switch, a filter cap, a filter grate, and/or any combination thereof. The housing having an inner container that is sized and configured to receive at least one of a battery pack, a circuit, an alarm and/or toggle switch. The housing having at least one opening on the body that the at least one opening extends through, and is sized and configured to receive a toggle switch. The filter media can comprise P95 filters, P100 filters, HEPA filters, HE filters and/or any combination thereof. The at least one light comprises a fluorescent lamp (CFL), an incandescent bulb, a halogen bulb, a light emitting diode (LED) bulb, an ultra-violet (UV) light, and/or any combination thereof.

In one embodiment, the PAPR kit comprises a neck-shoulder fitted PAPR kit. The neck-shoulder fitted PAPR kit comprises a hood assembly, a PAPR unit, and an at least one air hose. The PAPR kit may further comprise a battery charger kit. The PAPR kit, the PAPR unit and/or hood assembly may comprise a hood, and a head suspension frame. The PAPR kit, the PAPR unit and/or hood assembly may further comprise at least one light. The hood may comprise a rigid or a flexible hood. The hood may comprise a tight fitting hood or a loose fitting hood. The flexible hoods may comprise a head cover, a head cover with shroud, head cover with double shroud, a headcover sealed seam hood. The rigid hoods may comprise a helmet, a helmet with shroud and a helmet with a double shroud. The hood and/or the hood assembly may comprise a pathogen inactivation coating. The pathogen inactivation coating may comprise a germicidal, a solvent/detergent, a methylene blue coating, an amotosalen coating & UV light, a riboflavin coating & UV light, and/or any combination thereof. The PAPR unit comprises a blower motor, a battery pack and at least one filter media. At least a portion of the PAPR unit may be coupled and/or or disposed within the back region of the hood and/or hood assembly. At least a portion of the PAPR unit may be positioned within the back region of the hood and adjacent to the neck-shoulder region. The PAPR unit may further comprise at least one or more of a low voltage alarm, an air flow sensor, a toggle switch, a filter cap, a filter grate, and/or any combination thereof. The filter media can comprise P95 filters, P100 filters, HEPA filters, HE filters and/or any combination thereof. The hood assembly may further comprise a collar. The collar may be disposed within the neck region or adjacent to the neck region and/or may be adjustable from a loose position to a tightened position.

In another embodiment, the PAPR kit comprises a neck-shoulder fitted PAPR kit. The neck-shoulder fitted PAPR kit comprises a hood assembly and a PAPR unit. The PAPR kit may further comprise a battery charger kit. The PAPR kit, the PAPR unit and/or hood assembly may comprise a hood, a head suspension frame, and a collar. The PAPR kit, the PAPR unit and/or the hood assembly may further comprise at least one light. The hood may comprise a rigid or a flexible hood. The hood may comprise a tight-fitting hood or a loose fitting hood. The flexible hoods may comprise a head cover, a head cover with shroud, head cover with double shroud, a headcover sealed seam hood. The rigid hoods may comprise a helmet, a helmet with shroud and a helmet with a double shroud. The hood and/or the hood assembly may comprise a pathogen inactivation coating. The pathogen inactivation coating may comprise a germicidal, a solvent/detergent, a methylene blue coating, an amotosalen coating & UV light, a riboflavin coating & UV light, and/or any combination thereof. The PAPR unit comprises a blower motor, a battery pack and at least one filter media. At least a portion of the PAPR unit may be coupled and/or or disposed within the back region of the hood and/or hood assembly. At least a portion of the PAPR unit may be positioned within the back region of the hood and adjacent to the neck-shoulder region. The PAPR unit may further comprise at least one or more of a low voltage alarm, an air flow sensor, a toggle switch, a filter cap, a filter grate, and/or any combination thereof. The filter media can comprise P95 filters, P100 filters, HEPA filters, HE filters and/or any combination thereof. The at least one light comprises a fluorescent lamp (CFL), an incandescent bulb, a halogen bulb, a light emitting diode (LED) bulb, an ultra-violet (UV) light, and/or any combination thereof. The collar may comprise one or more elongated members, each of the one or more elongated members are “U-shaped” or generally “U-shaped.” Each of the one or more elongated members having an arc or arch shaped configuration with a channel running along the entire length of the one or more elongated members. The channel being sized and configured to receive one or more battery packs, the battery packs comprise one or more batteries. The batteries may be rechargeable or non-rechargeable. The collar may be adjustable and/or removably connected to the hood and/or hood assembly. The removably connected may comprise mechanical connections known in the art, including Velcro, quick release, snaps, pockets within the hood that are sized and configured for the collar to be disposed in. The collar may be flexible, elastic, and/or flexible and elastic to allow the users to manipulate it by bending, stretching and/or flexing it for proper fitting around head, neck and/or top of shoulders and releasing it to allow it to return to its original position. The collar may be flexible and bent to hold its position to allow the users to bend the collar, flex but maintain the position for proper fitting around head, neck and/or top of shoulders.

In another embodiment, the PAPR kit comprises a neck-shoulder fitted PAPR kit. The neck-shoulder fitted PAPR kit comprises a hood assembly, a PAPR unit, and at least one air hose. The PAPR kit may further comprise a battery charger kit. The hood assembly may comprise a hood, a hood plate and a head suspension frame. The PAPR kit, the PAPR unit and/or hood assembly may further comprise at least one light. The hood may comprise a rigid or a flexible hood. The hood may comprise a tight-fitting hood or a loose-fitting hood. The flexible hoods may comprise a head cover, a head cover with shroud, head cover with double shroud, a headcover sealed seam hood. The rigid hoods may comprise a helmet, a helmet with shroud and a helmet with a double shroud. The hood and/or the hood assembly may comprise a pathogen inactivation coating. The pathogen inactivation coating may comprise a germicidal, a solvent/detergent, a methylene blue coating, an amotosalen coating & UV light, a riboflavin coating & UV light, and/or any combination thereof. The PAPR unit comprises at least one battery pack, a blower motor, a filter cap and at least two filter media. At least a portion of the PAPR unit may be coupled and/or or disposed within the back region of the hood and/or hood assembly. At least a portion of the PAPR unit may be positioned within the back region of the hood and adjacent to the neck-shoulder region. Alternatively, at least a portion of the PAPR unit may be positioned within the back region of the shroud of the hood and/or hood assembly. The PAPR unit may further comprise at least one or more of a low voltage alarm, an air flow sensor, a toggle switch, a filter grate, and/or any combination thereof. The at least two filter medias can comprise P95 filters, P100 filters, HEPA filters, HE filters and/or any combination thereof. The battery packs positioned in the front region of the hood and/or hood assembly. The battery packs may be positioned in the front region of the hood and/or hood assembly adjacent to the neck-shoulder region.

In another embodiment, the PAPR kit comprises a surgical PAPR kit. The surgical PAPR kit comprising: a PAPR unit, the PAPR unit including a filter media and a blower, the blower including a blower air flow rate, at least a portion of the PAPR unit may be coupled and/or or disposed within the back region of the hood and/or hood assembly. At least a portion of the PAPR unit may be positioned within the back region of the hood and/or hood assembly, and/or adjacent to the neck-shoulder region; a hood assembly, the hood assembly comprising a hood, and at least one light, the at least one light being disposed in a front region of the hood, the hood including at least one opening, the at least one opening being disposed within the top region of the hood, the at least one opening being sized and configured to have an exhaust flow rate less than the blower air flow rate to maintain positive pressure within the hood assembly; a head suspension frame, at least a portion of the head suspension frame being disposed within the hood, the head suspension frame comprising a ring frame and a base, the base having a first opening and a second opening, the frame being adjustable around the circumference of a user's head, at least a portion of the PAPR unit being coupled to the second opening of the base; a flexible hose, the flexible hose comprising a first end and a second end, the first end being coupled to the first opening of the base, the second end being free and positioned adjacent to the front region of the hood; a collar, the collar being adjustable around a user's neck to create a sealed barrier or a substantially sealed barrier; and a battery pack, the battery pack being disposed into at least a portion of the collar or at least a portion of the head suspension frame. The surgical PAPR kit may further comprise a standard surgical gown, the hood assembly may mate, or be coupled or contact the surgical gown, and/or be tucked into a portion of the standard surgical gown. Alternatively, the surgical PAPR kit may further comprise a one-piece suit, the one-piece suit comprising a hood assembly and a gown.

In another embodiment, the PAPR kit comprises an alternate embodiment of a surgical PAPR kit. The surgical PAPR kit comprising: a PAPR unit, the PAPR unit including a filter media and a blower, the blower including a blower air flow rate, at least a portion of the PAPR unit may be coupled and/or or disposed within the back region of the hood and/or hood assembly. At least a portion of the PAPR unit may be positioned within the back region of the hood and adjacent to the neck-shoulder region; a hood assembly, the hood assembly comprising a hood, and at least one light, the at least one light being disposed in a front region of the hood, the hood including at least one opening, the at least one opening being disposed within the top region of the hood, the at least one opening being sized and configured to have an exhaust flow rate less than the blower air flow rate to maintain positive pressure within the hood assembly; a head suspension frame, at least a portion of the head suspension frame being disposed within the hood, the head suspension frame comprising a ring frame and a base, the base having a first opening and a second opening, the frame being adjustable around the circumference of a user's head, at least a portion of the PAPR unit being coupled to the second opening of the base, the first opening being free to provide the air flow rate of the blower; and a collar, the collar being adjustable around a user's neck to create a sealed barrier or a substantially sealed barrier; and a battery pack, the battery pack being disposed into a portion of the collar or a portion of the head suspension frame. The surgical PAPR kit may further comprise a standard surgical gown, the hood assembly may mate, or be coupled or contact the surgical gown, and/or be tucked into a portion of the standard surgical gown. Alternatively, the surgical PAPR kit may further comprise a one-piece suit, the one-piece suit comprising a hood assembly and a gown.

In another embodiment, the PAPR kit comprise an alternate embodiment of a dual filter PAPR kit. The dual filter PAPR kit comprising: a PAPR unit, the PAPR unit including a first filter media, a second filter media and a blower, the first filter media having a first axis and the second filter media having a second axis, the first axis of the first filter media is spaced apart and parallel to the second axis of the second filter media; a hood, the hood comprising a shroud; a head suspension frame, the head suspension frame being adjustable around the circumference of a user's head, the head suspension frame disposed within a top region of the hood; and a flexible hose, the flexible hose comprising a first end and a second end, the second end of the flexible hose being free and disposed near the front region of the hood. wherein the first filter media comprises the same or different filter media compared to second filter media. The PAPR unit is disposed within a back region of the hood. The PAPR unit is disposed within a back region of the hood and adjacent to the neck and shoulder region. The PAPR unit is disposed within a back region of the shroud of the hood. The dual filter PAPR kit may further comprise at least one or more of a hood plate, an acoustic tube, a first alarm and a second alarm, and/or any combination thereof. The hood plate positioned adjacent to the head suspension frame. The acoustic tube comprising a first end and a second end, the first end of the acoustic tube is coupled to a portion of the blower, and the second end of the acoustic tube is coupled to a first end of the flexible tube. The acoustic tube comprises an open cell or a closed cell foam. The first alarm comprises at least one light, the at least one light disposed within the front region of the hood, the at least one light comprises an LED light. The second alarm comprises an air flow sensor, the air flow sensor comprises a flexible assembly, the flexible assembly is disposed within the flexible hose, at least a portion of the flexible assembly extends beyond the second end of the flexible hose. The at least a portion of the flexible assembly is movable from a first position to a second position, the first position being within the peripheral vision of a wearer to indicate low air flow or no air flow, and the second position being outside of the peripheral vision of the wearer to indicate sufficient air flow.

### DETAILED DESCRIPTION

Powered air-purifying respirators (PAPRs) continually supply positive air pressure to a respirator to maintain positive pressure in the respirator. PAPRs are generally used in military, industrial or hazardous environments to provide personal respiratory protection by preventing ambient air from entering the user's mask, helmet, or hood that may contain various respiratory hazards. Accordingly, the PAPR kit may be also used in a surgical setting to help safeguard against contamination, exposure to infectious bodily fluids, transfer of microorganisms or particulate matter to the clinician and the patient. Respiratory hazards might include harmful microorganisms, particulate matter, harmful gases and/or vapors, which are removed by passing the ambient air through the filter. More specifically, the respiratory hazards may include impermissible exposure limits of various chemicals (e.g., formaldehyde, ethylene oxide, glutaraldehyde, etc.), blood-borne pathogens, and/or any aerosol-transmissible diseases.

Typically, a powered air-purifying respirator includes a powered fan that forces ambient air through one or more filters for delivery to an inlet opening in the respirator. The fan and/or filter may be mounted on the hood or hood assembly, or in some cases, may be mounted on a belt or backpack and connected to the facemask through a hose and a fan. Power for the fan is desirably mounted remote from the facemask, but in some embodiments could be mounted on the mask itself.

The PAPR kit relates to an improvement to powered air filtering devices incorporating a hood assembly, including a hood or hood assembly (e.g., helmet, mask, etc.), a PAPR unit, and at least one hose. The PAPR unit may comprise a blower, a battery pack and at least one filter media with various particulate, gas or combination filters for respiratory protection as shown in various alternate embodiments disclosed herein. The improved PAPR kit minimizes the number of parts and interconnections, thereby reducing time for assembly and disassembly. Fewer parts with smoother design consistency that greatly facilitates ease, speed, and cost effectiveness due to eliminating decontamination between uses The advantage of using the improved PAPR kit is that the kit may be disposable and does not place additional personnel at risk during repair, replacement, cleaning and/or disinfection.

Furthermore, the PAPR kit and/or PAPR units may be manufactured with no disconnects or assembly, no maintenance ports or service windows. The PAPR kit/PAPR unit is a self-contained, ready to use unit with a choice to not recharge the batteries, repair, replace, clean and/or disinfect the PAPR unit. In one embodiment, it would be advantageous to provide a PAPR kit incorporating a hood assembly, including a helmet, a mask or a hood (i.e., a PAPR Hood) in a disposable and/or single-use format.

PAPR Kits

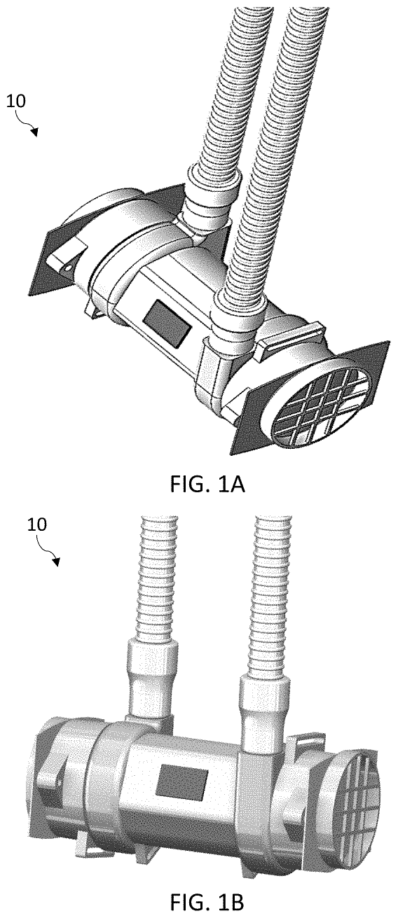

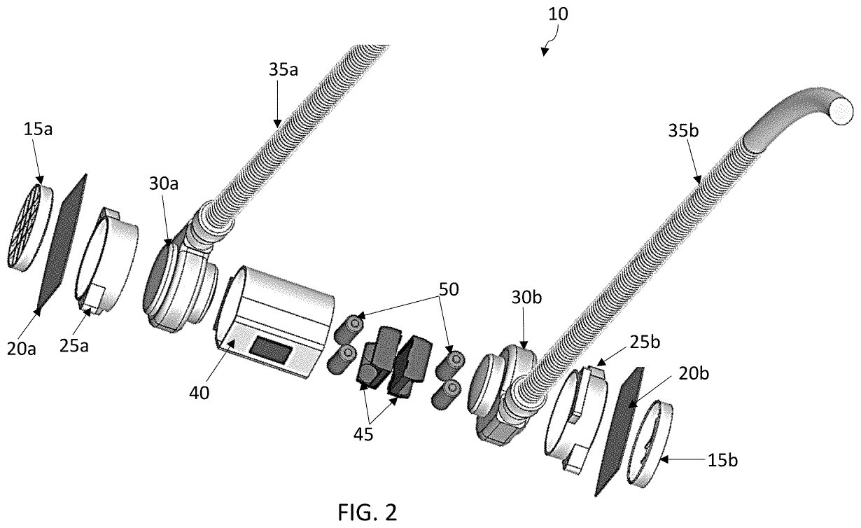

FIGS. 1A-1B and 2 depicts isometric views and an exploded view of one embodiment of a PAPR unit 10. The PAPR kit comprises a hood and/or hood assembly (not shown), a belt (not shown), a PAPR unit 10. The PAPR kit may further comprise a battery charger kit (not shown). At least a portion of the PAPR unit 10 coupled to at least a portion of the belt. The PAPR unit 10 comprises a plurality of filter grates or caps 15a, 15b, a plurality of filter medias 20a, 20b, a plurality of filter bases 25a, a plurality of blower motors 30a, 30b, a plurality of hoses 35a, 35b, a plurality of battery pack assemblies 45,50, at least one housing 40. The PAPR unit 10 may further comprise a low voltage alarm (not shown), an air flow sensor (not shown), a toggle switch (not shown), at least one indicator light (not shown) and/or any combination thereof. The housing 40 having a first end, a second end, and an inner container that extends through the first end to the second end. The inner container is sized and configured to receive at least one of battery pack assemblies 45,50, a circuit (not shown), the low voltage alarm, and/or the toggle switch. At least a second end of each of the plurality of blower motors 30a,30b is coupled or secured to a first end and second end of the housing 40. At least a first end of the each of the plurality of blower motors 30a,30b is coupled or engages with a second end of each of the plurality of filter cap bases 25a,25b. A first end of each of the plurality of filter cap bases 25a,25b is coupled to or engages with each of the plurality of filter medias 20a,20b. Each of the plurality of filter caps 15a,15b is disposed over each of the plurality of filter medias 20a,20b, and secured to each of the plurality of filter cap bases 25a,25b. Each of the plurality of blower motors 30a,30b is positioned opposite direction or opposing directions. A second end of each of the plurality of hoses 35a, 35b is coupled to each of the plurality of blower motors 30a, 30b. The first end of each of the plurality of hoses 35a, 35b is coupled to the hood.

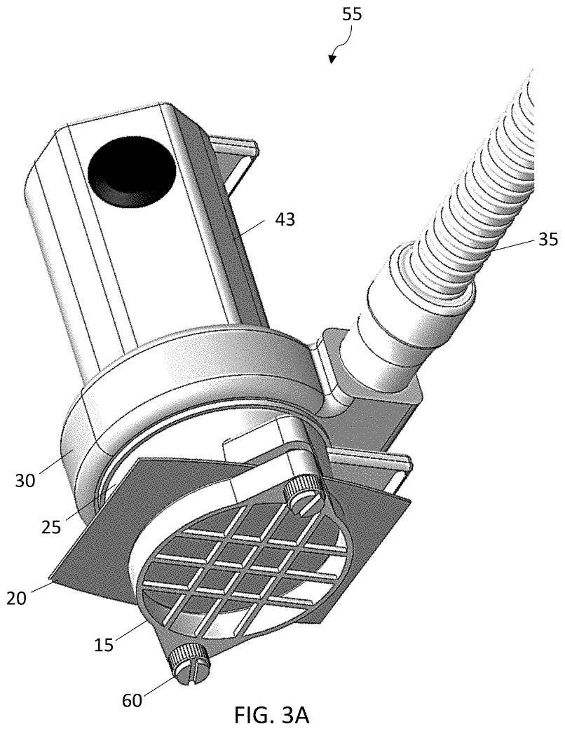

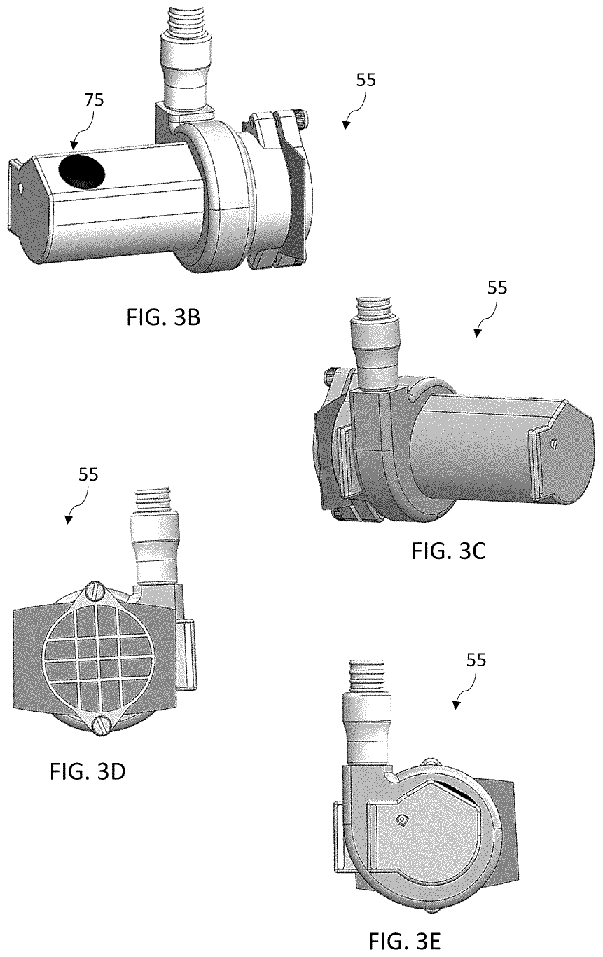

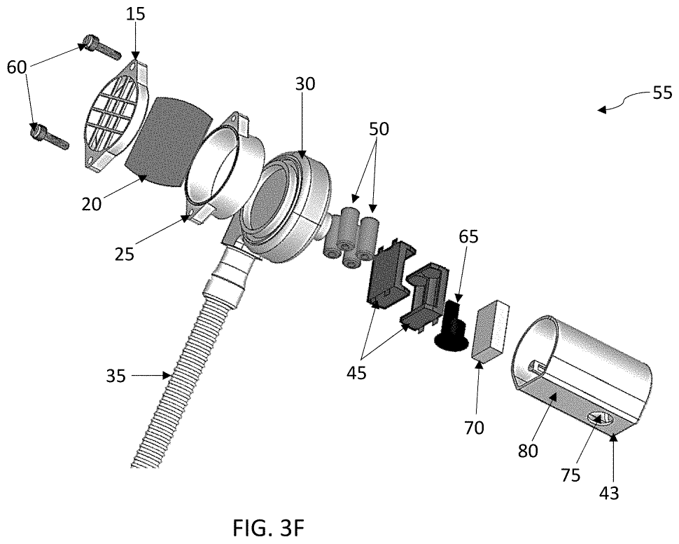

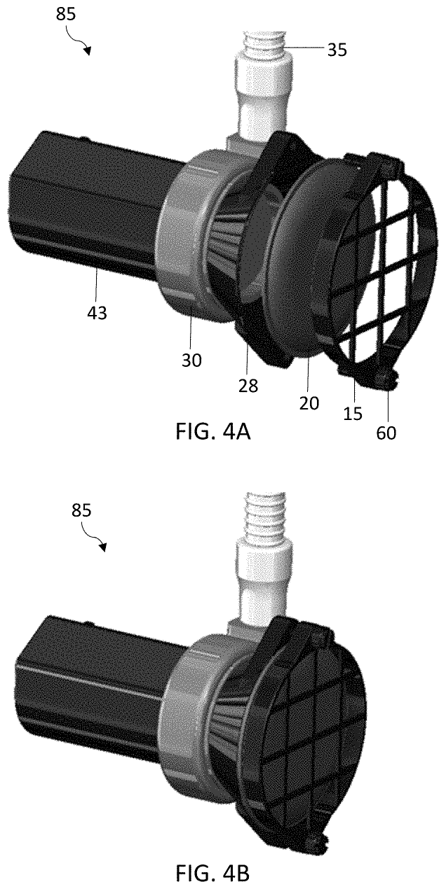

FIGS. 3A-3F and 4A-4B depicts isometric views and an exploded view of an alternate embodiments of a PAPR units 55,85. The PAPR kit (not shown) comprises a hood and/or hood assembly (not shown), a belt (not shown), a PAPR unit 55, 85. The PAPR kit may further comprise a battery charger kit (not shown). At least a portion of the PAPR unit 55, 85 coupled to at least a portion of the belt. The PAPR unit 55, 85 comprises a at least one filter cap or grate 15, at least one filter medias 20, at least one filter base 25, 28, at least one blower motor 30, at least one hose 35, at least one battery pack assembly 45,50, and/or at least one housing 43. The PAPR unit 55,85 may further comprise a low voltage alarm (not shown), an air flow sensor (not shown), a toggle switch (not shown), at least one indicator light (not shown) and/or any combination thereof. The housing 43 having a first end and a second end, the second end including a bottom surface to define an inner container. The inner container is sized and configured to receive at least one of battery pack assemblies 45,50, a circuit (not shown), the low voltage alarm, and/or the toggle switch. At least a second end of at least one blower motor 30 is coupled or secured to a first end and second end of the housing 43. At least a first end of the at least one blower motor 30 is coupled or engages with a second end of the at least one filter cap base 25, 28. A first end of the at least one filter cap base 25, 28 is coupled to or engages with the at least one filter media 20. The at least one filter caps or grates 15 is disposed over the at least one filter medias 20, and secured to the at least one filter cap base 25, 28. A second end of the at least one hoses 35 is coupled to the at least one blower motor 30. The first end of the at least one hose 35 is coupled to the hood.

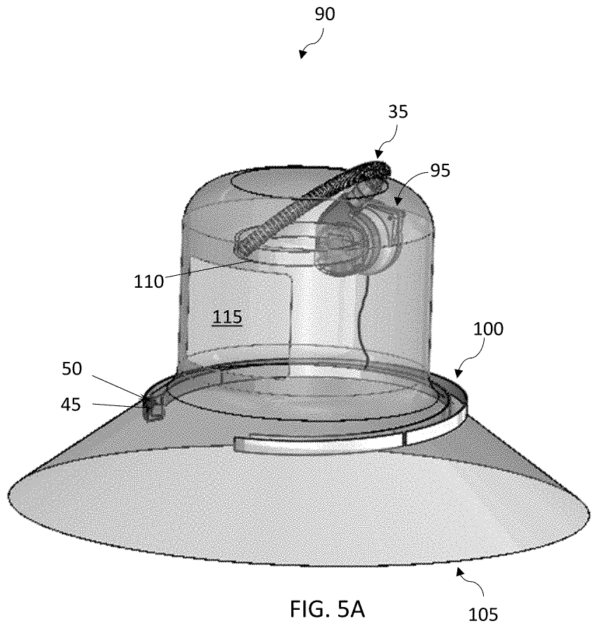

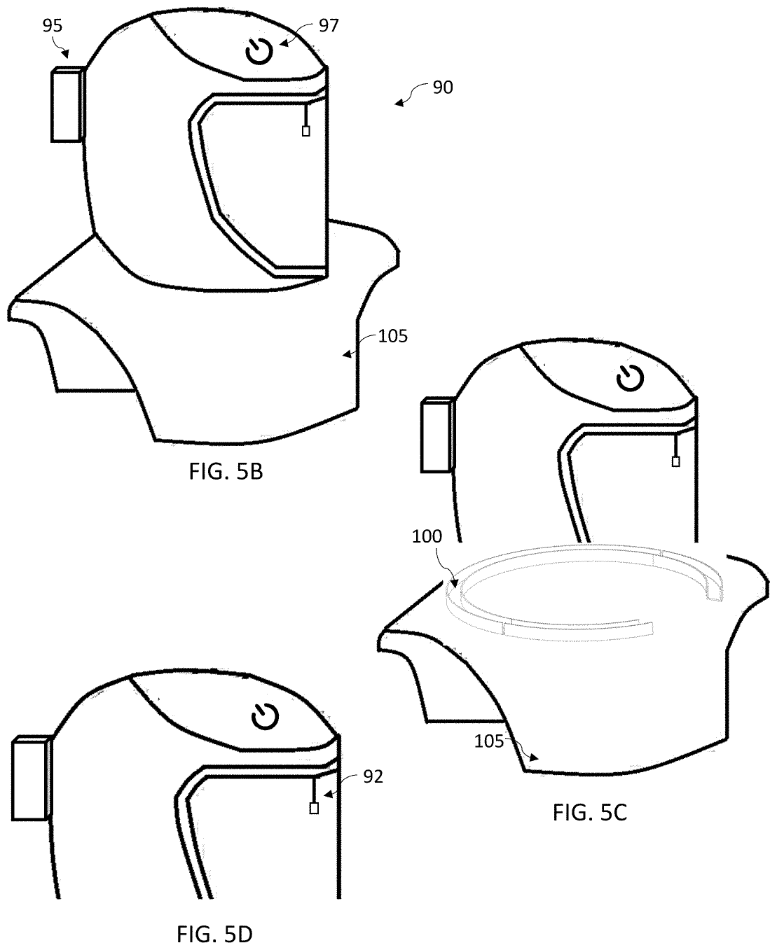

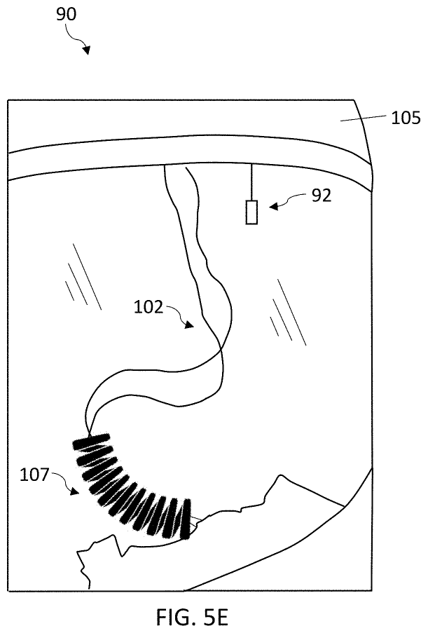

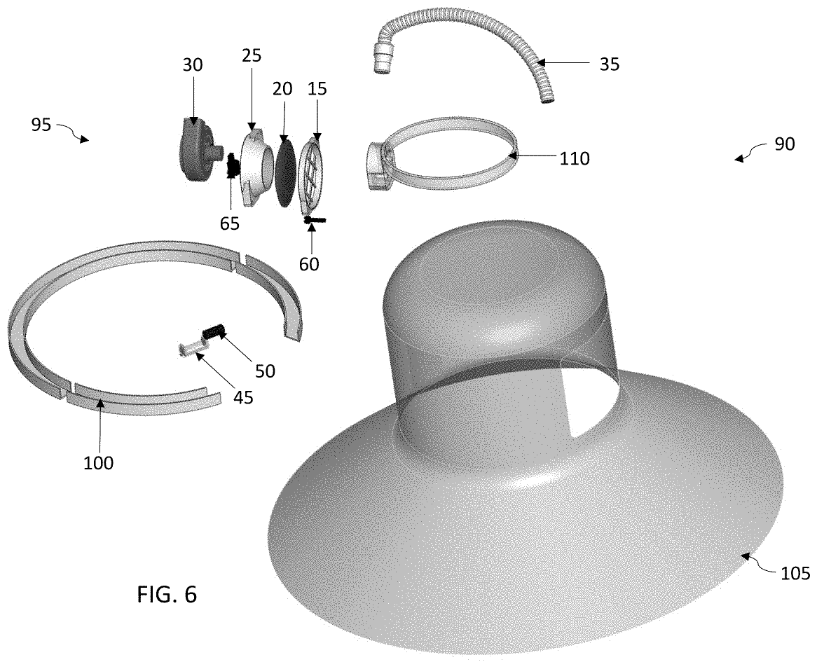

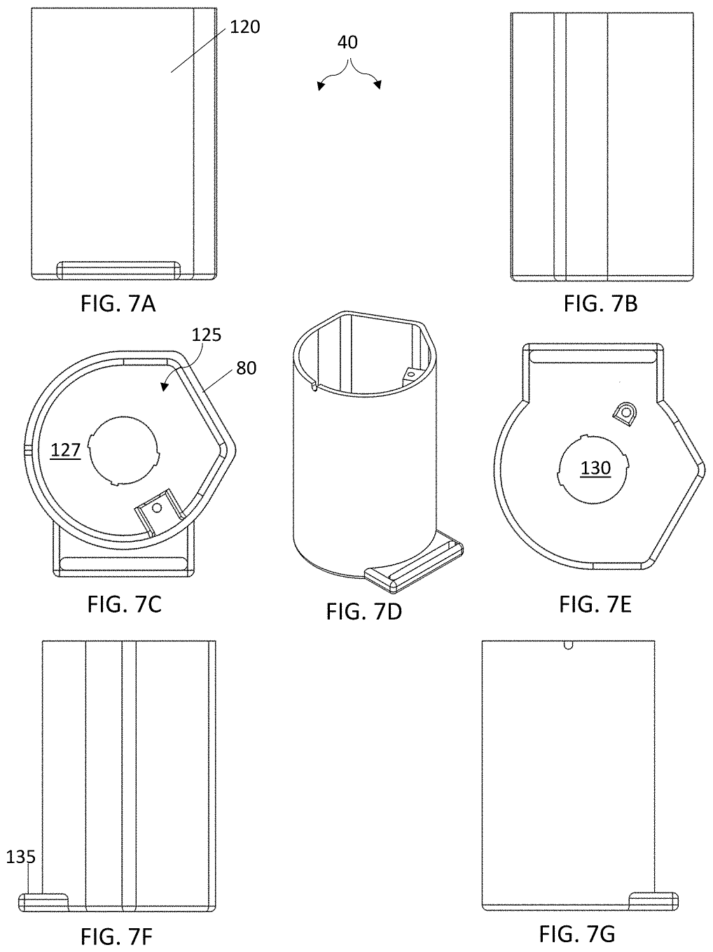

FIGS. 5A-5E and FIG. 6 depict various views of an alternate embodiment of a PAPR kit 90. The PAPR kit 90 comprises a hood and/or hood assembly 105, a PAPR unit 95 a head suspension frame 110, and a collar 100. The PAPR kit may further comprise a battery charger kit (not shown) and at least one light 92. The head suspension frame 110 disposed in the top region of the hood 105, the head suspension frame 110 disposed adjacent or proximate to an inner surface of the hood in the top region. The head suspension frame 110 sized and configured to be disposed on a wearer's head, the head suspension frame 110 can be adjustable. The hood 105 may comprise an opening and a viewing window. The opening is disposed in the back region of the hood 105 and the viewing window is disposed in the front region of the hood 105. The at least a portion of the head suspension frame 110 is coincident and/or aligned with the opening. Accordingly, at least a portion of the head suspension frame 110 may extend into the opening.

The PAPR unit 95 and/or a portion of the PAPR unit 93 is coupled or secured to the head suspension frame 110. The PAPR unit 95 comprises a at least one filter cap or grate 15, at least one filter medias 20, at least one filter base 25, 28, at least one blower motor 30, a at least one hose 35, at least one battery pack assembly 45,50 The PAPR unit 95 may further comprise a low voltage alarm (not shown), an air flow sensor (not shown), a toggle switch (not shown), at least one indicator light (not shown) and/or any combination thereof. At least a first end of the at least one blower motor 30 is coupled or engages with a second end of the at least one filter cap base 25, 25. A first end of the at least one filter cap base 25, 28 is coupled to or engages with the at least one filter media 20. The at least one filter caps or grates 15 is disposed over the at least one filter media 20, and secured to the at least one filter cap bases 25, 28. A second end of the at least one hose 35 is coupled to the at least one blower motor 30. The at least one hose 35 is disposed within the top region or crown of the hood 105. More specifically, the at least one hose 35 is disposed within the top region and/or crown of the hood 105 and adjacent or proximate to an inner surface of the hood 105.

The collar 100 is disposed onto the wearer's neck, the collar 100 is disposed onto the wearer's neck adjacent or proximate to an inner surface of the hood 105 and/or adjacent or proximate to an outer surface of the hood 105. The collar 105 may be adjustable and/or removably connected to the hood 105. The collar 105 may comprise a “U-shape” or generally “U-shape.” The collar 105 may comprise one or more elongated members having an arc or arch shaped configuration with a channel running along the entire length of the one or more elongated members. The channel being sized and configured to receive one or more battery pack assemblies 45,50. The collar 105 may comprise a material, the material may be flexible, elastic, and/or flexible and elastic to allow the users to manipulate it by bending, stretching and/or flexing it for proper fitting around head, neck and/or top of shoulders and releasing it to allow it to return to its original position. The collar 105 may be flexible and bent to hold its position to allow the users to bend or flex the collar 105, but maintain the position for proper fitting around head, neck and/or top of shoulders.

The at least one light 92 may be disposed within the hood 105, at least a portion of the at least one light 92 may disposed in the top region of the hood, at least a portion of the at least one light may be disposed within the hood in a top region or crown region. At least a portion of the at least one light 92 extends into a portion of the viewing window of the hood. The at least a portion of the at least one light 92 is proximate or adjacent to a wearer's eyes, and/or proximate or adjacent to a wearer's eyes within the wearer's peripheral vision.

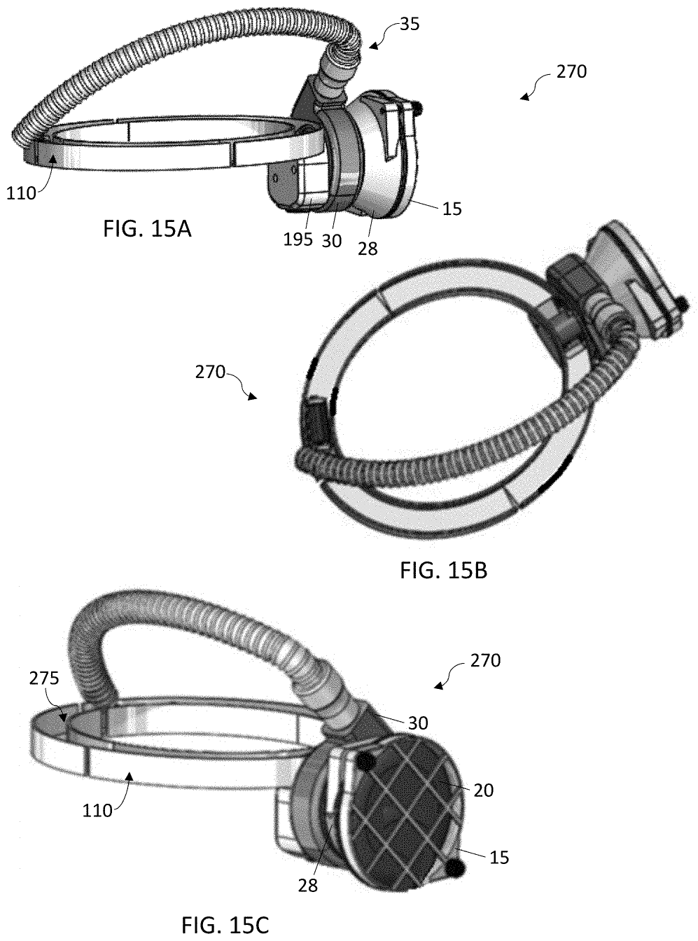

FIGS. 15A-15C depict various views of an alternate embodiment of a PAPR kit. The PAPR kit comprises a hood and/or hood assembly 105, a PAPR unit 270, and/or a head suspension frame 110. The PAPR kit may further comprise a battery charger kit (not shown) and at least one light 92. The head suspension frame 110 disposed in the top region of the hood 105, the head suspension frame 110 disposed adjacent or proximate to an inner surface of the hood in the top region. The head suspension frame 110 sized and configured to be disposed on a wearer's head, the head suspension frame 110 can be adjustable. The head suspension frame 110 may further comprise a channel, the channel may be sized and configured to receive one or more battery pack assemblies 45,50. The hood 105 may comprise an opening and a viewing window. The opening is disposed in the back region of the hood 105 and the viewing window is disposed in the front region of the hood 105. The at least a portion of the head suspension frame 110 is coincident and/or aligned with the opening. Accordingly, at least a portion of the head suspension frame 110 may extend into the opening.

The PAPR unit 270 and/or a portion of the PAPR unit 270 is coupled or secured to the head suspension frame 110. The PAPR unit 270 comprises a at least one filter cap or grate 15, at least one filter medias 20, at least one filter base 25, 28, at least one blower motor 30, a at least one hose 35, at least one battery pack assembly 45,50 The PAPR unit 270 may further comprise a low voltage alarm (not shown), an air flow sensor (not shown), a toggle switch (not shown), at least one indicator light (not shown) and/or any combination thereof. At least a first end of the at least one blower motor 30 is coupled or engages with a second end of the at least one filter cap base 25,28. A first end of the at least one filter cap base 25,28 is coupled to or engages with the at least one filter media 20. The at least one filter caps or grates 15 is disposed over the at least one filter media 20, and secured to the at least one filter cap bases 25,28. A second end of the at least one hose 35 is coupled to the at least one blower motor 30. The at least one hose 35 is disposed within and/or extends through the top region or crown of the hood 105. More specifically, the at least one hose 35 is disposed within the top region and/or crown of the hood 105 and/or extends through the top region or crown of the hood 105. The at least one hose may be adjacent or proximate to an inner surface of the hood 105. The first end of the at least one hose 35 is free, the first end positioned adjacent or proximate to the viewing window of the hood and/or the eyes of a wearer.

The at least one light 92 may be disposed within the hood 105, at least a portion of the at least one light 92 may disposed in the top region of the hood, at least a portion of the at least one light may be disposed within the hood in a top region or crown region. At least a portion of the at least one light 92 extends into a portion of the viewing window of the hood. The at least a portion of the at least one light 92 is proximate or adjacent to a wearer's eyes, and/or proximate or adjacent to a wearer's eyes within the wearer's peripheral vision.

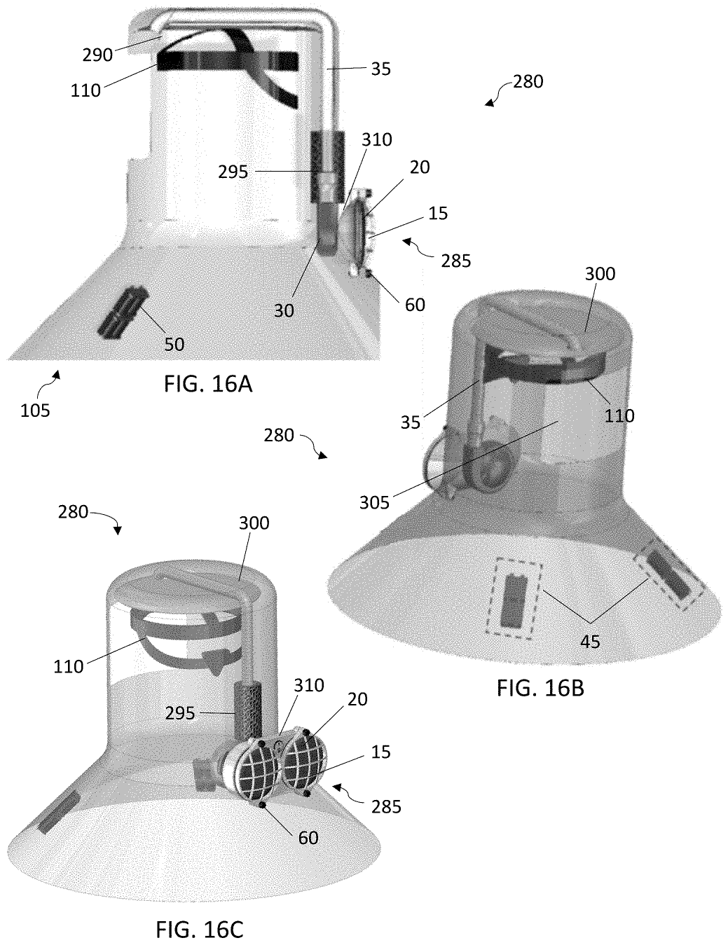

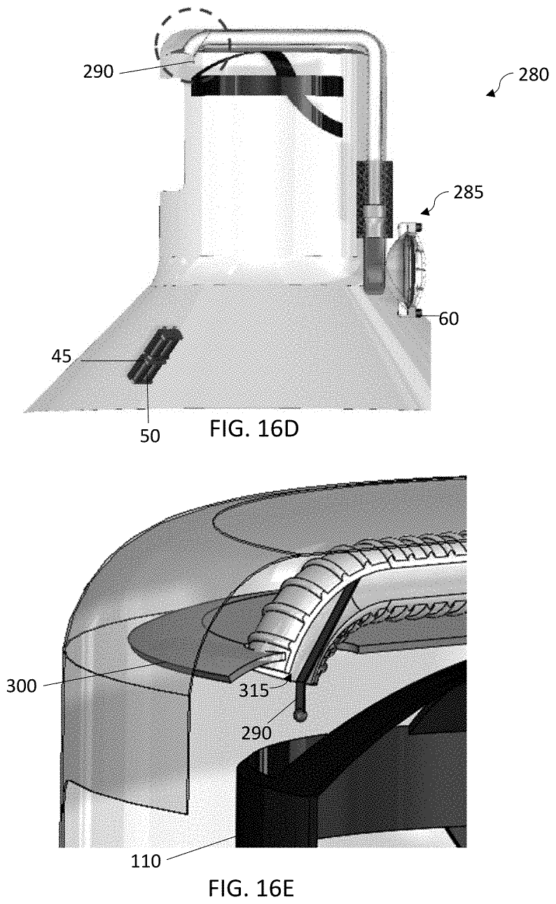

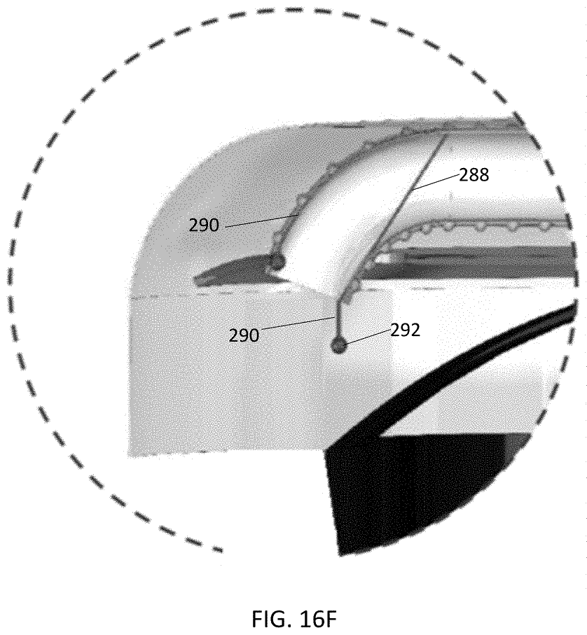

FIGS. 16A-16F, 17A-17D and 22A-22B depict various views of an alternate embodiment of a PAPR kit 280, 380. The PAPR kit 280, 380 comprises a hood and/or hood assembly 105, a PAPR unit 285, 385, and/or a head suspension frame 110. The PAPR kit 280, 380 may further comprise a battery charger kit (not shown), at least one light 92, at least one head or hood plate 300, at least one acoustic tube 295, at least one air flow sensor 290 and/or any combination thereof. The head suspension frame 110 disposed in the top region of the hood 105, the head suspension frame 110 disposed adjacent or proximate to an inner surface of the hood in the top region. The head suspension frame 110 sized and configured to be disposed on a wearer's head, the head suspension frame 110 can be adjustable. The hood 105 may comprise an opening and a viewing window 305. The opening is disposed in the back region of the hood 105 or occipital region, and/or the opening is disposed in the back region of the hood and adjacent to the neck-shoulder region. The opening is spaced apart or offset from the head suspension frame 110. The viewing window 305 is disposed in the front region of the hood 105. The at least a portion of the head suspension frame 110 is offset from the opening and/or spaced apart from the opening. The head or hood plate 300 disposed in the top region of the hood 105, the head or hood plate 300 disposed adjacent or proximate to an inner surface of the hood 105 in the top region. The head or hood plate 300 is spaced apart and parallel to the head suspension frame 110. The head or hood plate 300 having a shape, the shape may be oval, circular and/or a polygon. The shape may further comprise an arch or concave shape.

The PAPR unit 285, 385 and/or a portion of the PAPR unit 285, 385 mates or engages with the hood opening and/or the at least a portion of the PAPR unit 285, 385 extends through the hood opening. The PAPR unit 285, 385 comprises a one or more filter cap grates 15, 15a, 15b, one or more filter medias 20, 20a, 20b, at least one filter base 310, at least one blower motor 30, at least one hose 35, one or more battery pack assemblies 45,50. The PAPR unit 285 may further comprise a low voltage alarm (not shown), an air flow sensor 290, a toggle switch 330, at least one indicator light (not shown) and/or any combination thereof. At least a first end of the at least one blower motor 30 is coupled or engages with a second end of the at least one filter cap base 310. A first end of the at least one filter cap base 310 is coupled to or engages with one or more filter medias 20, 20a, 20b. The one or more filter caps or grates 15, 15a, 15b is disposed over the one or more filter medias 20, 20a, 20b, and secured to the at least one filter cap base 310. A second end of the at least one hose 35 is coupled to the at least one blower motor 30. The at least one hose 35 is disposed within the top region or crown of the hood 105 and is disposed over the head plate 300. More specifically, the at least one hose 35 is extends from the back region of the hood 105 towards the top region and/or crown of the hood 105 over the head plate 300. The first end of the at least one hose 35 is free, the first end is adjacent or proximate to the viewing window of the hood and/or the eyes of a wearer. The acoustic tube 295 may enclose or encase a portion of the at least one hose 35 and/or the second end of the at least one hose 35. Alternatively, the acoustic tube 295 may enclose the entirety of the at least one hose 35.

The at least one light 92 may be disposed within the hood 105, at least a portion of the at least one light 92 may disposed in the top region of the hood, at least a portion of the at least one light may be disposed within the hood in a top region or crown region. At least a portion of the at least one light 92 extends into a portion of the viewing window of the hood. The at least a portion of the at least one light 92 is proximate or adjacent to a wearer's eyes, and/or proximate or adjacent to a wearer's eyes within the wearer's peripheral vision.

The at least one air flow sensor 290 may comprise a flexible assembly (e.g., a telltale). The flexible assembly comprises a string or fabric that moves and changes direction with the wind or airflow. At least a portion of air flow sensor 290 is disposed within the at least one hose 35 and/or within an inner diameter 315 of the at least one hose 35. At least a portion of the air flow sensor 290 extends beyond the second end or first end of the at least one flexible hose 35. Alternatively, the at least at least a portion of the air flow sensor 290 is movable from a first position to a second position, the first position being within the peripheral vision of a wearer to indicate low air flow or no air flow, and the second position being outside of the peripheral vision of the wearer to indicate sufficient air flow.

The one or more battery pack assemblies 45,50 may be disposed in the front region of the hood 105. The one or more battery pack assemblies 45,50 may be disposed on the front region of the shroud of the hood 105. The one or more battery pack assemblies 45,50 may be disposed on the front region on an external surface of the shroud of the hood 105. Alternatively, the one or more battery pack assemblies 45,50 may be disposed on the front region on an inner surface of the shroud of the hood 105.

In another embodiment, the PAPR kit comprises a surgical PAPR kit (not shown). The surgical PAPR kit comprising: a PAPR unit, the PAPR unit including a filter media and a blower, the blower including a blower air flow rate, at least a portion of the PAPR unit may be coupled and/or or disposed within the back region of the hood and/or hood assembly. At least a portion of the PAPR unit may be positioned within the back region of the hood and/or hood assembly, and/or adjacent to the neck-shoulder region; a hood assembly, the hood assembly comprising a hood, and at least one light, the at least one light being disposed in a front region of the hood, the hood including at least one opening, the at least one opening being disposed within the top region of the hood, the at least one opening being sized and configured to have an exhaust flow rate less than the blower air flow rate to maintain positive pressure within the hood assembly; a head suspension frame, at least a portion of the head suspension frame being disposed within the hood, the head suspension frame comprising a ring frame and a base, the base having a first opening and a second opening, the frame being adjustable around the circumference of a user's head, at least a portion of the PAPR unit being coupled to the second opening of the base; a flexible hose, the flexible hose comprising a first end and a second end, the first end being coupled to the first opening of the base, the second end being free and positioned adjacent to the front region of the hood; a collar, the collar being adjustable around a user's neck to create a sealed barrier or a substantially sealed barrier; and a battery pack, the battery pack being disposed into at least a portion of the collar or at least a portion of the head suspension frame. The surgical PAPR kit may further comprise a standard surgical gown, the hood assembly may mate, or be coupled or contact the surgical gown, and/or be tucked into a portion of the standard surgical gown. Alternatively, the surgical PAPR kit may further comprise a one-piece suit, the one-piece suit comprising a hood assembly and a gown.

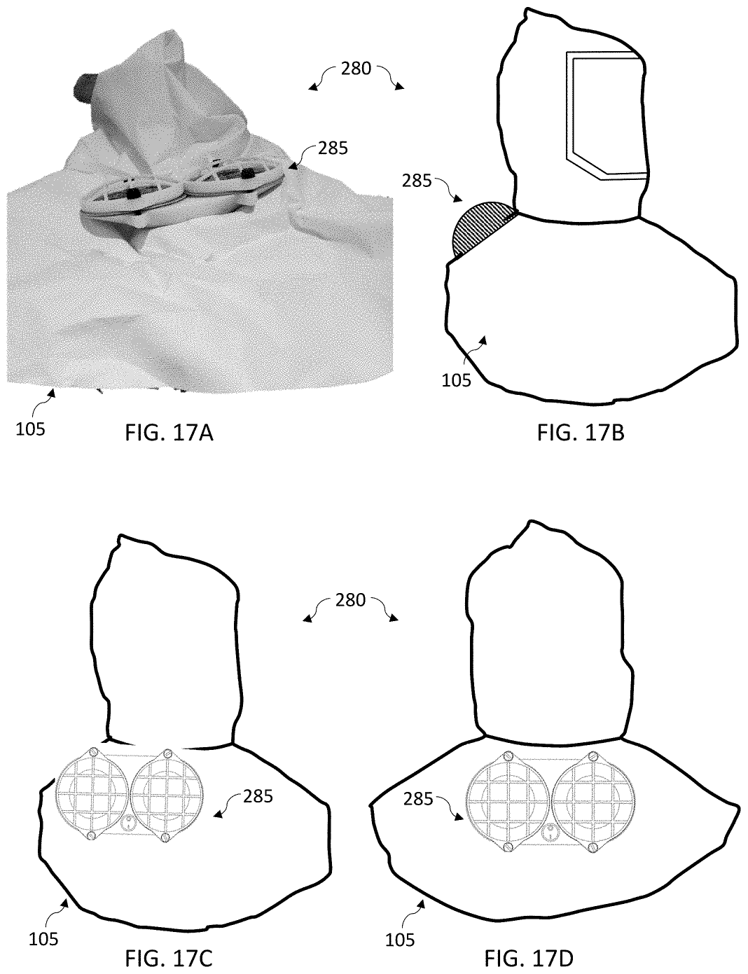

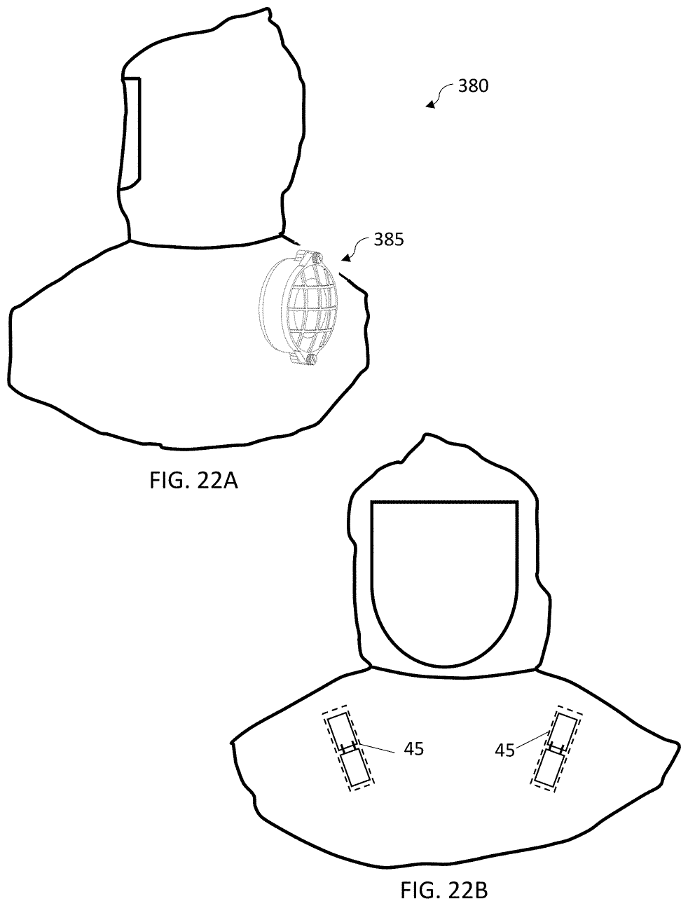

In various embodiments, the PAPR unit may be disposed, coupled or secured to various regions of the wearer. In one embodiment, the PAPR unit is disposed within a back region of the hood (FIGS. 5A-5E, and 15A-15C). The PAPR unit is disposed within a back region of the hood and adjacent to the neck and shoulder region (FIGS. 16A-16C, 17A-17D and 22A-22B). The PAPR unit is disposed within a back region of the hood adjacent to the neck and should region on the shroud of the hood (FIGS. 17A-17D and 22A-22B). The PAPR unit is disposed, coupled and/or secured adjacent and/or within the waist region of a wearer (FIGS. 1A-1B, 2, 3A-3F, 4A-4B). In another embodiment, the PAPR unit is disposed on the front region of the hood and/or hood assembly (not shown). The PAPR unit may be disposed on the front region of the hood and/or the front region of a shroud of the hood or the hood assembly (not shown). The PAPR unit may be disposed on an outer surface of a shroud of the hood or hood assembly (not shown). Furthermore, the PAPR unit may comprise a weight less than traditional PAPR units, the weight may include 0.75 lb to 1.5 lbs, which is a 33% to 50% reduction of weight compared to traditional PAPR units.

PAPR Kit Components

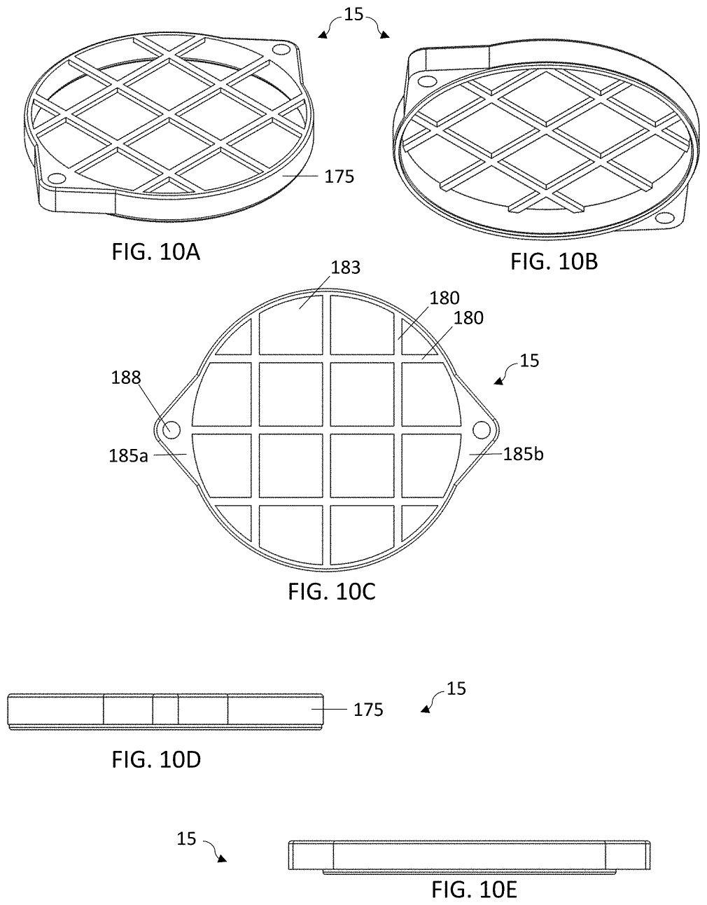

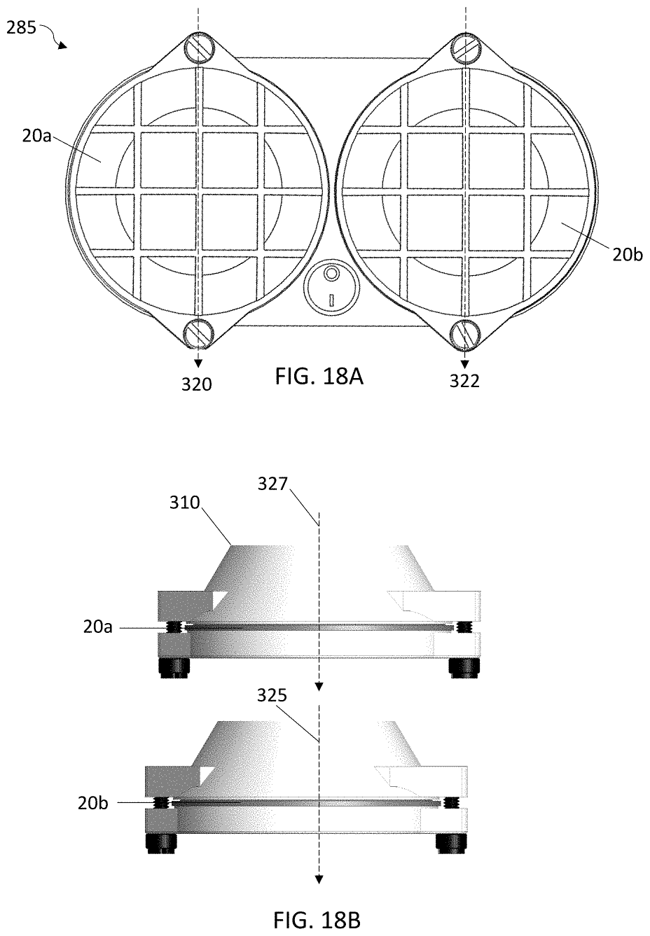

FIGS. 2, 3F, 4A-4B, 10A-10E, 18A-18B and 19A-19B depict various views of different embodiments of a filter grate 15, 15a, 15b. The filtration grate 15, 15a, 15b comprises an ring 175, and a plurality of struts 180 that are interconnected to form a mesh or matrix with a plurality of openings 183. The plurality of interconnected struts 180 being disposed within an inner diameter of the ring 175. The filtration grate 15, 15a, 15c includes a shape, the shape comprises a generally circle or generally oval, and/or a generally cylindrical shape. The filtration cap 15, 15a, 15b further comprises one or more tabs 185a, 185b that extend outward perpendicular to the outer body or outer diameter of the filtration cap ring 175. The tabs 185a, 185b includes at least one opening 188, the at least one opening 188 is sized and configured to receive a connection mechanism 60. In an alternative embodiment, the PAPR unit and/or PAPR kit may comprise a plurality of filtration grates 15, 15a, 15b as shown in FIGS. 18A-18B. The plurality of filtration grates 15, 15a, 15b are spaced apart and positioned colinear to each other. Alternatively, the plurality of filtration grates 15, 15a, 15b are spaced apart and positioned parallel to each other. The plurality of filtration grates 15, 15a, 15b disposed over a plurality of filtration media 20, 20a, 20b. Accordingly, the PAPR unit and/or PAPR kit may comprise a first filtration grate 15a and a second filtration grate 15b. At least a portion of the first filtration grate 15a may be disposed over a portion of a first filtration media 20a, and at least a second portion of the second filtration grate 15a may be disposed over a second filtration media 20b.

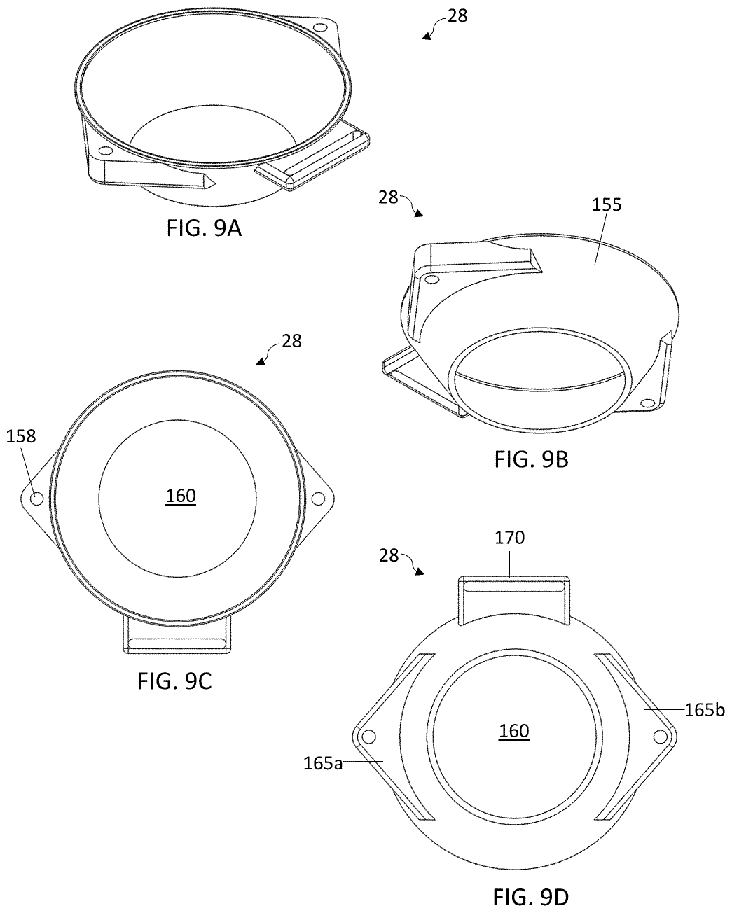

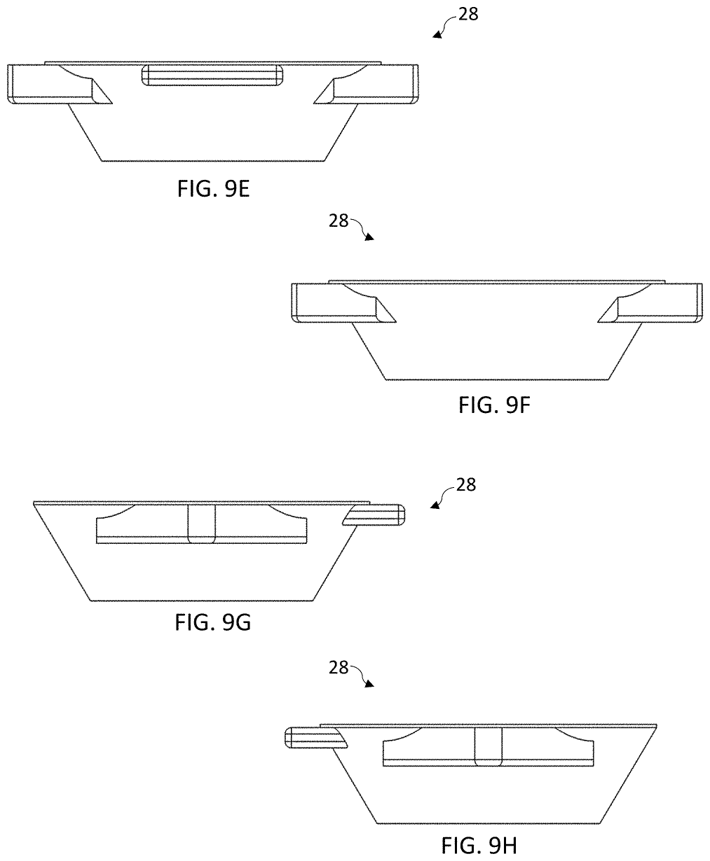

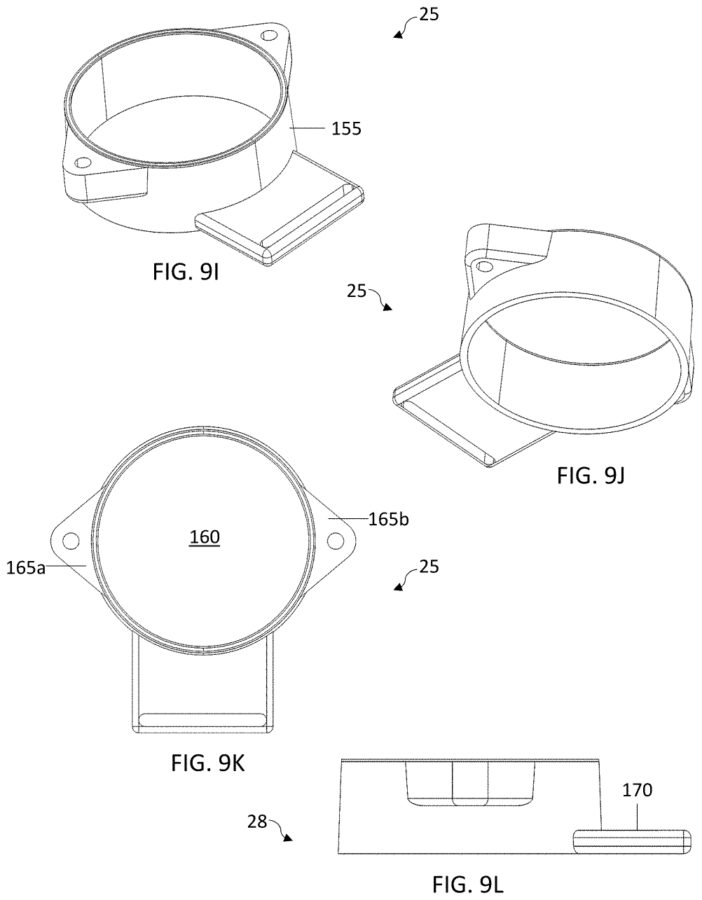

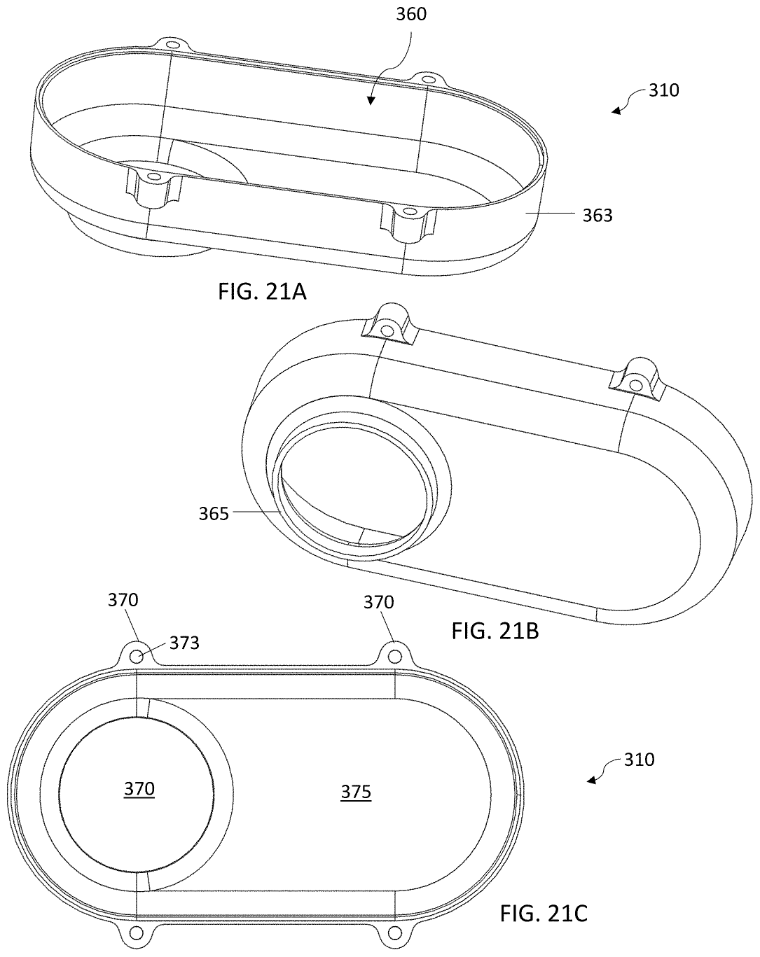

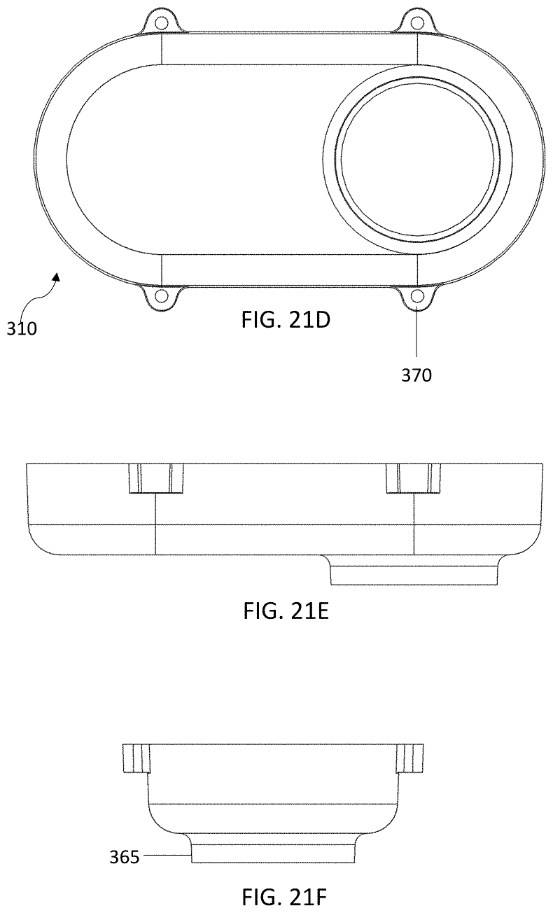

FIGS. 2, 3F, 4A-4B, 9A-9H, 9I-9L, FIGS. 18A-18B, 19A-19B and 21A-21F depict various embodiments of a filtration cap or filtration base 25, 28, 310. The filtration base 25, 28 may comprise a body 155, the body 155 including a first end and a second end, and an opening 160. The opening 160 extending through the first end through the second end. The filtration base 25, 28 comprise a shape, the shape includes a circle, an oval and/or a cylindrical shape. Alternatively, the filtration base 25, 28 shape may further comprise a generally shaped circle, oval and/or cylindrical. In another embodiment, the body 155 of the filtration base 25, 28 may comprise a cone or frustum shape. The opening 160 being sized and configured to receive at least one filtration media. The filtration base 25, 28 may further comprise a plurality of tabs 165a, 165b, 170. The plurality of tabs 165a, 165b, 170 may extend outwardly from the outer diameter of the body 155 and/or may extend perpendicularly from the outer diameter of the body 155. The plurality of tabs 165a, 165b, 170 may comprise a tab opening 158, the tab opening 158 is sized and configured to receive a secure mechanism 60.

In another embodiment, the filtration base 310 may comprise a body 363, the body 363 including a first end and a second end. The first end or second end may comprise a recess 360 and/or a bottom surface 375. The bottom surface 375 may comprise a vent opening 370. The first or second end may further comprise an exhaust vent or a flange 365. The exhaust vent or flange 365 surrounds the vent opening 370 and/or is concentric with the inlet. The exhaust vent or flange 365 extends outwardly from the first end or second end. The body 363 may comprise a shape, the shape may include an oval, a rounded rectangle, or an ellipse. The shape may further include a generally oval shape, a generally rounded rectangle or a generally elliptical shape. The body 363 may further comprise tabs 370, the tabs 370 may comprise at least one opening 373, the at least one opening 373 is sized and configured to receive a securement mechanism 60.

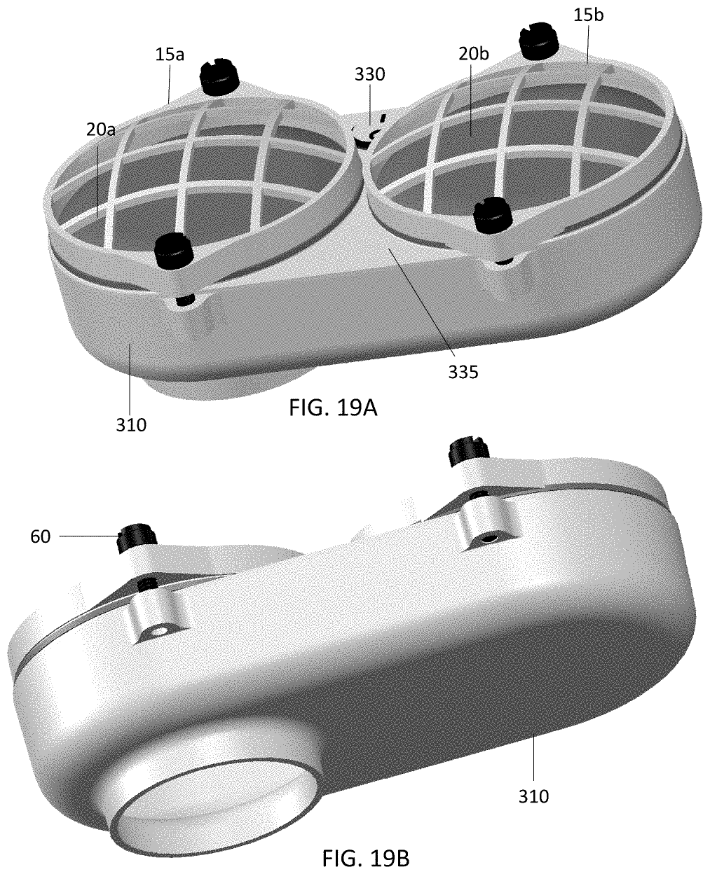

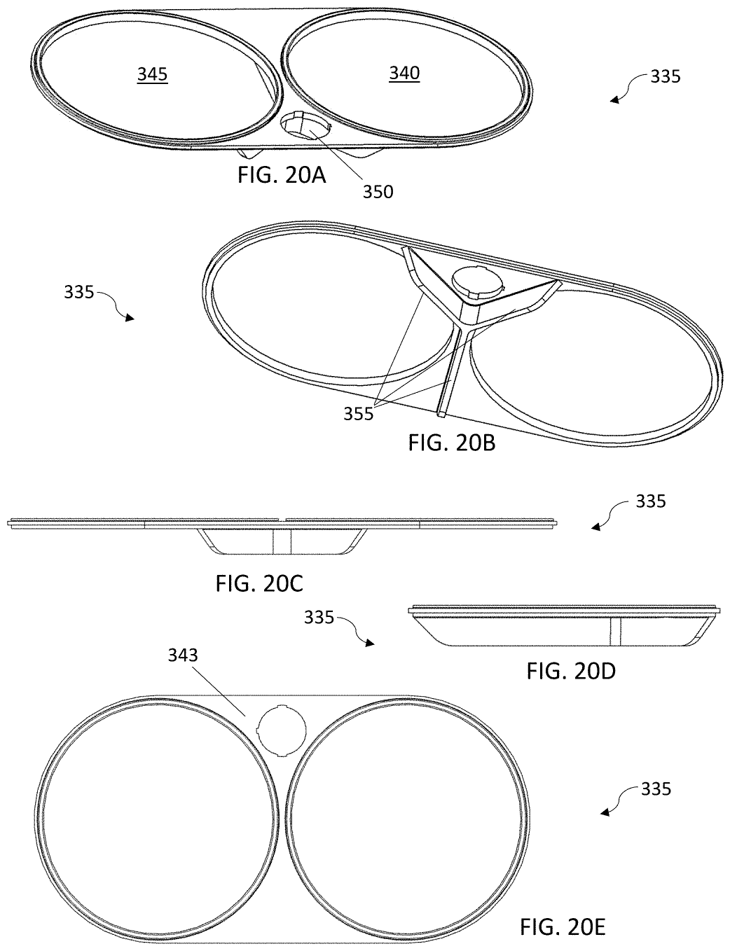

FIGS. 19A-19B and 20A-20E depicts various views of one embodiment of a filtration plate or filtration cap plate 335. The filter plate 335 comprising a body 343. The body 343 may comprise an elongated rounded rectangle shape, an elongated circle and/or an elongated ellipse. The body 343 having a top surface and a bottom surface. The body 343 having at least three openings 340,343,350 that extend from the top surface through the bottom surface. The body 343 sized and configured to be disposed within the recess 360 of the filtration base 310. At least two 340, 343 of the three openings are sized and configured to receive a portion of a plurality of filtration medias. The at least one or remaining opening 350 is sized and configured to receive a portion of a toggle switch (or “on/off switch”). At least openings 340,343 are spaced apart and parallel to each other.

Alternatively, the body 343 having a first opening 345, a second opening 350 and a third opening 350. The first 345 and second opening 350 extending from the top surface through the bottom surface. The first opening 345 sized and configured to receive a portion of a first filtration media, and the second opening 350 sized and configured to receive a portion of a second filtration media. The third opening 350 is sized and configured to receive a portion of a toggle switch.

The bottom surface of the body 373 comprises a plurality of walls 355. Each of the plurality of walls 355 are oriented at a plurality of angles and are coupled at least one end of each of the plurality of walls 355 to create an interconnection joint. The angle may comprise a range of 30 to 75 degrees. The plurality of angles and/or each of the plurality of angles may be the same or different. The plurality of walls 355 extend outwardly from the bottom surface of the filter plate body 343 and/or the plurality of walls 355 extend perpendicularly from the bottom surface of the filter plate body 343. The filtration plate 335 is disposed between the filter grates 15a, 15b and the filter base 310.

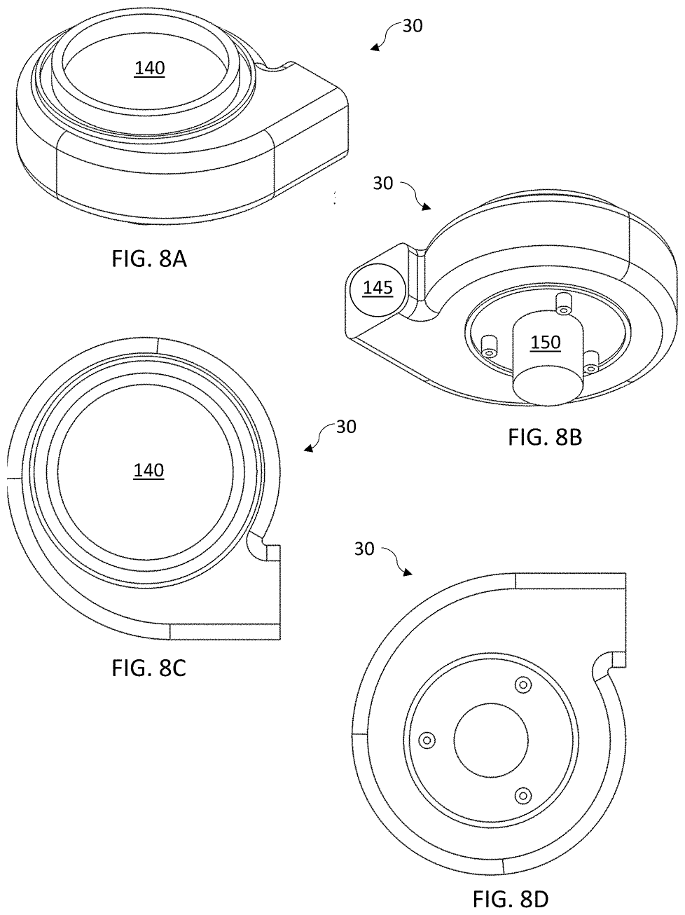

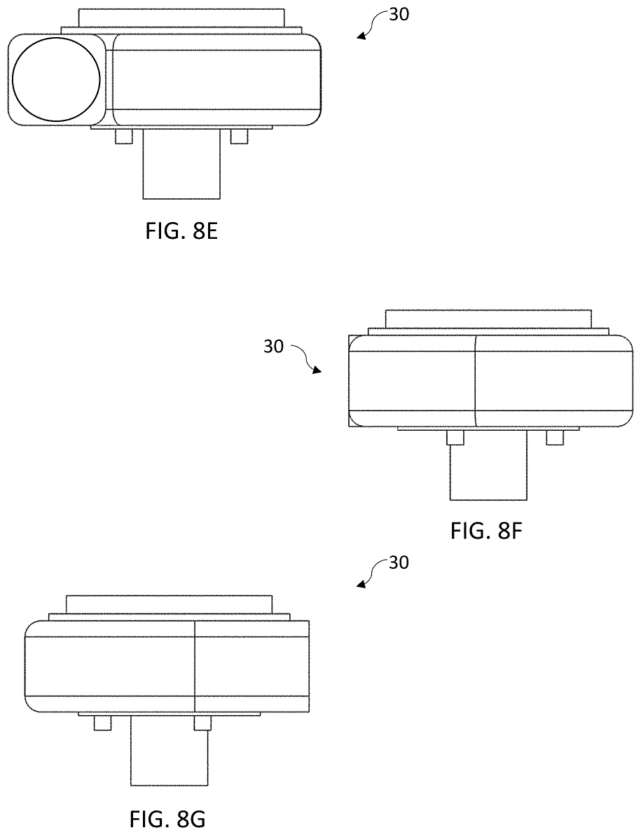

FIGS. 8A-8G depicts various views of one embodiment of a blower motor 30.

The blower motor 30 may comprise an inexpensive, disposable battery-powered blower or fan unit that is significantly lighter than traditional blower motors 30. The blower motor 30 comprises at least one air flow speed setting or air flow rate setting. Alternatively, the blower motor 30 may comprise two or more air flow speed settings. The air flow rate may comprise a range of 115 L/min to 510 L/min. More specifically, the air flow rate may comprise a range of 115 L/min to 250 L/min. The air flow rate may comprise an initial flow rate that occurs when the PAPR unit is “turned on or powered on” or activated and a steady flow rate or sustained flow rate that occurs after several hours of use, the initial flow rate is greater than the steady flow rate. In another embodiment, the sustained flow rate must be equal to or greater than a user's inhalation rate or a user's respiratory minute volume during rest, light activities, moderate exercise and/or severe or significant exercise and/or any combination thereof. The blower motor 30 may comprise rated voltage of 5 V to a 12 V DC and may comprise a weight of 2 ounces to 6 ounces.

The blower motor 30 may comprise a first end and a second end. The second end and/or first end comprises a recess 140 or a longitudinal member 150. The blower motor 30 further comprises an air outlet 145. At least one end of the at least one tube or the plurality of tubes may be coupled to the air outlet 145. In an alternative embodiment, the PAPR unit may comprise a first blower and a second blower. The first blower having a first air flow rate and the second blower having a second air flow rate. The first air flow rate may comprise the same or different air flow rate than the second air flow rate.

FIG. 2, FIGS. 3A-3F, and 4A-4B depicts an isometric view of one embodiment of a housing 40, 43. In one embodiment, the housing 40,43 comprises a body, the body 120 includes a shape, the shape comprises a cylindrical shape or generally cylindrical shape. The body 120 may be hollow. The hollow body 120 having an opening 125 extending from a first end through the second end. At least a portion of the body 120 comprises a flat surface or planar surface 80. The flat or planar surface 80 may be used as a mounting surface. The flat or planar surface 80 may include a switch opening 75, the switch opening 75 is sized and configured to receive a toggle switch. Alternatively, the body 120 having an inner container that includes an opening 125 on a first end and a second end. In another embodiment, the housing 40, 43 may comprise a body, the body comprising a first end and a second end, the first end includes an opening 125, and the second end includes a bottom surface 127 to form an inner container. The bottom surface 127 comprising at least one opening 130. The opening or the inner container 125 is sized and configured to receive at least a portion of the battery pack, circuit, switch, at least one light, at least one alarm and/or any combination thereof. The second end of the body may further comprise a tab 135, the tab 135 extends perpendicularly from an outer surface of the body.

FIG. 2, 3F, 5A, 6, 16A-16E, 22B depicts various embodiments of a battery pack or battery pack assembly 45, 50. The battery pack assembly 45, 50 may comprise a small, light, inexpensive and/or disposable battery pack comprising one or more batteries. The battery pack assembly 45, 50 may be fully integrated, and self-contained with the PAPR unit to eliminate or prevent the ability to clean, repair, or replace. The battery pack 45, 50 may be removably connected from the PAPR unit to allow repair, replacement, and/or disinfection if desired. The removably connected battery pack 45, 50 may be removed and placed onto the battery charger kit (not shown) and the user replaces the discharged battery pack with a fully charged replacement battery pack. The battery pack 45, 50 may comprise an economy battery pack (run time approximately 4 to 6 hours), a medium capacity battery pack (run time approximately 6 to 8 hours), and a high-capacity battery pack (run time approximately 8 to 12 or 8 to 16 hours).

In alternative embodiments, the PAPR kit and/or PAPR unit may comprise a plurality of battery pack assemblies 45,50 and/or one or more battery pack assemblies 45, 50. The plurality of battery packs 45, 50 and/or each of the plurality of battery packs 45,50 may have the same charging voltage and/or amperage. The plurality of battery packs 45,50 and/or each of the plurality of battery packs 45,50 may have different charging voltage and/or amperage. The plurality of battery packs 45,50 is used to power a plurality of blower motors. Alternatively, the PAPR kit and/or PAPR unit may comprise a first battery pack and/or a second battery pack. The first battery pack is coupled to the first blower motor and the second battery pack is coupled to the second blower. Accordingly, the first batter pack and the second battery packs are coupled to both first and second blowers to power both blowers. The battery pack or battery pack assembly 45, 50 comprises one or more batteries 45 and a battery housing 50.

The battery pack assembly 45,50, the plurality of battery packs 45,50, the first battery pack and/or the second battery back may be disposed in a variety of regions and/or locations. The battery pack 45,50, the plurality of battery packs 45,50, the first battery pack and/or the second battery back may be disposed within the housing. The battery pack 45,50, the plurality of battery packs 45,50, the first battery pack and/or the second battery back may be disposed within a portion of a collar. The battery pack 45,50, the plurality of battery packs 45,50, the first battery pack and/or the second battery back may be disposed within a portion of a head suspension frame. The battery pack 45,50, the plurality of battery packs 45,50, the first battery pack and/or the second battery back may be disposed onto the hood. The battery pack 45,50, the plurality of battery packs 45,50, the first battery pack and/or the second battery back may be disposed onto a shroud of the hood. The battery pack 45,50, the plurality of battery packs 45,50, the first battery pack and/or the second battery back may be disposed onto a front region of the shroud of the hood. The battery pack 45,50, the plurality of battery packs 45,50, the first battery pack and/or the second battery back may be disposed onto an inner surface within the front region of the shroud of the hood, the inner surface facing towards the body or torso of a wearer. The plurality of battery packs 45,50, the first battery pack and/or the second battery back may be spaced apart and disposed onto an inner surface within the front region of the shroud of the hood, the inner surface facing towards the body or torso of a wearer. The inner surface of the shroud may comprise a plurality of pockets, a first pocket and/or a second pocket that are spaced apart, and/or sized and configured to receive a portion of the plurality of battery packs, a first battery pack and/or a second battery pack.

In various embodiments, the PAPR kit may comprise a stand-alone battery charger kit (not shown) and/or an integrated battery charger kit (not shown). The stand-alone battery charger kit enables the user to use and recharge a broad spectrum of the latest rechargeable battery technologies quickly and efficiently. The battery charger kit may comprise selectable current for different charging speeds. The battery charger kit may be a separate unit that is plugged into an AC outlet, similar to battery charger kits known in the art. In another embodiment, the battery charger kit may comprise a solar battery charger kit that is also a separate unit. The stand-alone solar battery charger kit comprises a solar panel (from 1.5 volts to 12 volts) or a solar power bank, a USB port, and a battery pack container sized to receive batteries that require charging. Alternatively, the solar battery charger kit may be integrated unit within the hood assembly. The integrated solar battery charger kit may comprise a one or more solar cells that matches or substantially matches the voltage and/or current of the rechargeable batteries and charge the batteries without power cords; one or more solar cells may be disposed onto the hood or hood assembly. The DC voltage and/or amperage would be directly connected to the rechargeable battery pack that is disposed within the hood and/or hood assembly to increase life/discharge cycle use and/or extend the life/discharge cycle of the battery. The integrated solar battery charger kit may comprise circuitry known in the art to help regulate and/or amplify the DC voltage and/or amperage.

FIGS. 1A-1B, 2, 3A-3F, 4A-4B, 6, 15A-15C, 16A-16C, 18A, 22A-22B depict various views of different embodiments of one or more filtration medias 20, 20a, 20b and/or at least one filtration media 20, 20a, 20b. The at least one filter media 20, 20a, 20b may be a “universal” filter media 20, 20a, 20b that can include a wide variety of filtration media (i.e., including, but not limited to, approved filtration media such as P95, N95, KN95 designations or higher) and/or available “emergency filtration” media such as filter components, fabric materials, cotton wadding and/or other filtering materials. The filtration media 20, 20a, 20b comprises a PM 2.5 filter, a P95 filter, an N95 filter, a KN95 filter, a P100 filter, a HE (high efficiency) filter, a HEPA filter, and/or any combination thereof. The HE filters may comprise a NIOSH approved 3M H E filter. In some alternative embodiments (not shown), the filter holder may include a second set of “bars” on an inside of the filter basket (i.e., on the inside of the lower filter portion), such as where some type of frangible filtration medium (like a handful of cotton balls or the like) is being used or where a “weaker” filtration medium that might tear under the vacuum pressure from the pump is being utilized. The filtration media 20, 20a, 20b may be fully integrated in a self-contained unit to prevent repair, replacement. The filtration media 20, 20a, 20b may be removably connected from the PAPR unit to allow repair, replacement, and/or disinfection. The filtration media 20, 20a, 20b may be disposed between the filtration grate 15, 15a, 15b and the filtration base 25, 28, 310. The filtration media 20, 20a, 20b may comprise a shape, the shape includes circular, oval, an ellipse or rounded rectangle shape.

In another embodiment, the PAPR kit may comprise a plurality of filter medias 20a, 20b. The plurality of filter medias 20a, 20b and/or each of the plurality of filter medias 20a, 20b may be the same or different. The plurality of filters 20a, 20b may be positioned adjacent to each other. The plurality of filters 20, 20b may be positioned parallel to each other. The plurality of filters 20a, 20b may be positioned colinear to each other. Alternatively, the PAPR kit may comprise a first filter media 20a and a second filter media 20b. The first filter media 20a is the same as the second filter media 20b. The first filter media 20a is different than the second filter media 20b. The first 20a and/or the second filter media 20b may comprise a P95 filter, an N95 filter, a KN95 filter, a P100 filter, an HE filter, a HEPA filter and/or any combination thereof. The first filter media 20a having a first axis 320 and the second filter media 20b having a second axis 322. The first axis 320, 327 of the first filter media 20a is positioned parallel to the second axis 322, 325 of the second filter media 20b. The first axis 320, 327 of the first filter media 20a is spaced apart and positioned parallel to the second axis 322, 325 of the second filter media 20b. Alternatively, the first axis 322, 327 of the first filter media 20a is positioned colinear to the second axis 322, 325 of the second filter media. The first axis 322, 327 of the first filter media 20a is spaced apart and positioned colinear to the second axis 322, 325 of the second filter media 20b.

FIG. 5B-5E depicts various views of one embodiment of the at least one light 92. The at least one light 92 and/or the at least one indicator light (not shown) comprises a fluorescent lamp (CFL), an incandescent bulb, a halogen bulb, a light emitting diode (LED) bulb, an ultra-violet (UV) light, and/or any combination thereof. The at least one light 92 comprises wires 102 and a light bulb. The at least one light 92 may further comprise flexible sheath 107, the flexible sheath 107 may be a coil or a tube. The flexible sheath 107 may assist with strain from flexing of the wires 102. At least a portion of the at least one light 92 may be positioned adjacent to the wearer's eyes and/or at least a portion of the at least one light 92 may be positioned adjacent to the wearer's eyes and within the wearer's peripheral vision. Alternatively, the at least one light 92 may be positioned adjacent or proximate to the viewing window of the hood 105. The at least one light 92 may be visualized by the wearer to indicate whether the PAPR unit is powered ON and indicate whether the batteries are running low due to dimming of the light.

The at least one light 92 and/or the indicator light may be “always off” and only triggered to “on” when used to inform the user of low air flow and/or low or depletion of batteries. The at least one light 92 and/or indicator light will may be “always on” when used to see at nighttime with a continuous light and “flash intermittently” to inform the user of low air flow and/or depletion of batteries. As the charging voltage and/or amperage of the batteries become low or fall below an acceptable charging threshold, the at least one light 92 may begin to dim and/or “flash intermittently.” In addition, the at least one light 92 may comprise a limited useful service life, the useful service life may comprise a range of 6 to 12 hours, 8 to 10 hours, 10 to 12 hours, and/or any combination thereof.

FIGS. 1A-1B, 2, 3A-3F, 4A-4B, 6, 15A-15C, 16A-16C, 18A, 22A-22B depict various views of different embodiments of the one or more hoses 35, 35a, 35b. The one or more hose tubing 35, 35a, 35b may comprise corrugations for flexibility and turns in tight areas. The hose tubing 35, 35a, 35b may comprise quick disconnects to the PAPR unit, the head suspension frame 110, the hood assembly and/or the hood 105. In one embodiment, the hose 35, 35a, 35b may comprise a first end and a second end, the first end or second end coupled to the PAPR unit or at least a portion of the PAPR unit, and the first or second end coupled to the hood and/or the hood assembly. Alternatively, the flexible hose 35, 35a, 35b may comprise a first end and a second end, the first end or second end is coupled to a portion of the head suspension frame 110 and/or coupled to the base 195 of the head suspension frame 110. The second end is free, the second end may extend over the crown of the user's top region of the head or over the crown member of the head suspension frame to direct the airflow on the hood's face shield for antifogging, comfort, and determination of airflow operation. In some embodiments, the powered air filtering device may incorporate a single supply air hose, while in alternative embodiments the unit may include a plurality of such hoses and/or other air supply arrangements. Exemplary embodiments of both single and dual supply air hoses are shown in the attached figures, as well as units incorporating a plurality of filter media. If desired, a unit may encompass two (2) Blower Fans for more outflow and/or power, which may alternatively encompass dual air inflow filters (i.e., an additional filter on the other end) for more volumetric air flow.

In various embodiments, the PAPR kit and/or PAPR unit may comprise a first hose and a second hose tubing. In one embodiment, the first hose may comprise a first end and a second end, the first end coupled to the PAPR unit, and the second end coupled to the hood and/or the hood assembly. Alternatively, the first hose may comprise a first end and a second end, the first end is coupled to a portion of the head suspension frame and/or coupled to the base of the head suspension frame. The second end is free, the second end may extend over the crown of the user's head or over the crown member of the head suspension frame to direct the airflow on the hood's face shield for antifogging, comfort, and determination of airflow operation. A portion of the second hose may be disposed within and/or coupled to the hood assembly and/or hood. A portion of the second hose may be disposed within the hood assembly and/or hood, and/or adjacent to the back region and/or top region of the hood and/or hood assembly. A portion of the second hose may be disposed on the top region and/or back region of the hood assembly and/or hood. The first hose may comprise an air supply hose, the second hose may comprise an air exhaust hose.

FIGS. 16A-16D depict various views of one embodiment of an acoustic tube 295. The acoustic tube 295 may be incorporated to decrease and/or dampen the noise (e.g., soundproofing or sound-absorbing) and vibrations created by the blower motor 30, 30a, 30b and/or the connection between the blower motor 30, 30a, 30b to the tube/flexible tube 35, 35a, 35b. The acoustic tube 295 may be positioned between a first end of the at least one hose 35, 35a, 35b and the second end of the at least one hose 35, 35a, 35b. Alternatively, at least a portion of the acoustic tube 295 may be positioned between a first end of the hose and the second end of the at least one hose 35, 35a, 35b.

The acoustic tube 295 may comprise a material and a shape. The shape comprises a hollow cylindrical tube. The shape may further comprise a wedge shape, a pyramid shape, an egg crate shape, a wave shape, a grid shape, a spade shape, a flat shape and/or any combination thereof. The material comprises a foam, wood wool, cellulose, fiberglass and/or any combination thereof. the foam includes an open cell foam and/or a closed cell foam. The open-cell foam comprises polyether, polyester or extruded melamine.

In another embodiment, the acoustic tube 295 may be added to create a new junction. To create a new junction, the PAPR unit comprises at least one acoustic tube 295 comprising a first end and a second end, a first hose and a second hose. Each of the first and second hose comprising a first end and a second end. The second end of the first hose coupled to the PAPR unit or blower, the first end of the first hose coupled to the second end of the at least one acoustic tube 295. The first end of the at least one acoustic tube 295 coupled to the second end of the second tube, the first end of the second tube is free and positioned adjacent to the front region of the hood and/or adjacent to the frontal region of the wearer. In another embodiment, the acoustic tube 295 may be added to insulate an existing blower-to-tube junction. To insulate an existing blower-to-tube junction, the acoustic tube 295 may be wrapped around the blower-to-flexible tube junction like a sleeve and/or the free end of the flexible tube. The acoustic tube having 295 a first end and a second end, the first end coupled to the blower to create a junction. At least a portion of the acoustic tube 295 disposed over at least a portion of the junction. At least a portion of the acoustic tube 295 disposed over the entire junction.

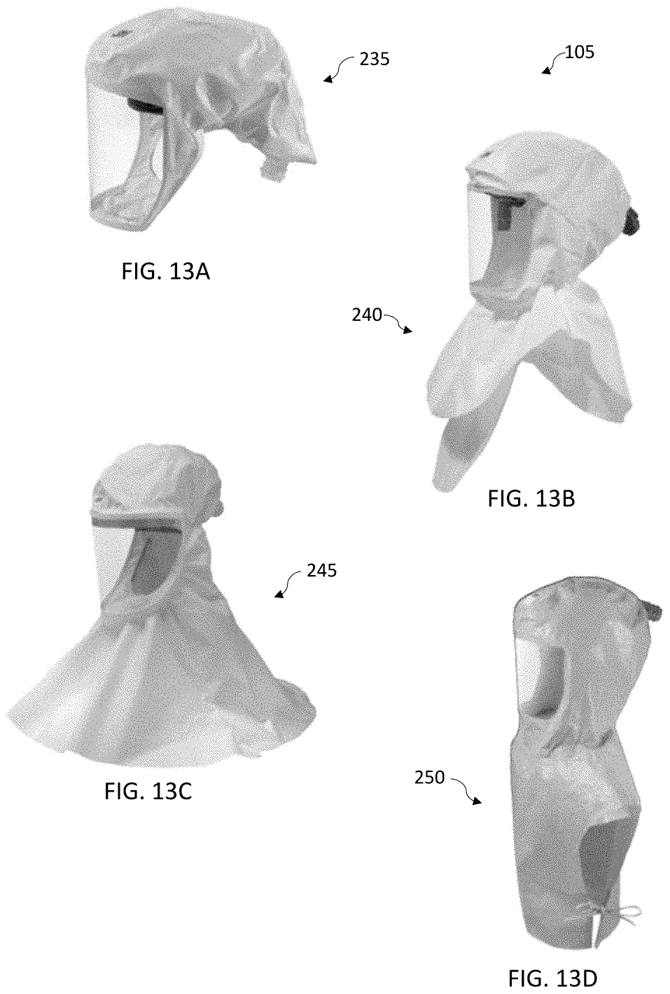

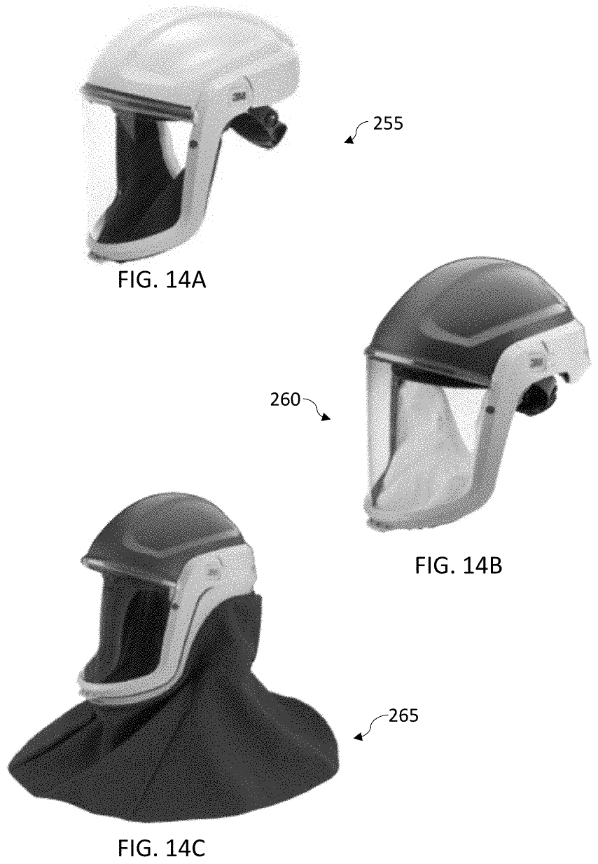

FIGS. 13A-13D, 14A-14C, 17A-17D and 22A-22B depict various views of different embodiments of a hood and/or hood assembly 105. The term “hood” or “hood assembly” is defined as a generic term to include the different embodiments of hoods disclosed herein. The hood assembly 105 may comprise hood, the hood includes a rigid hood, a flexible hood and/or an emergency hood. The “emergency” hood includes a clear plastic bag. The hood may comprise a tight-fitting hood or a loose-fitting hood. The flexible hoods may comprise a head cover 235, a head cover with shroud 240, head cover with double shroud 245, a headcover sealed seam hood 250. The shroud and/or double shroud having an outer surface and an inner surface. The outer surface facing towards the environment, the inner surface facing towards the body or torso of a wearer. The inner surface of the shroud may comprise a plurality of pockets, the plurality of pockets may be spaced apart. The plurality of pockets may be sized and configured to receive at least a portion of the plurality of battery packs. Alternatively, the inner surface of the shroud may comprise a first pocket and a second pocket, the first pocket is spaced apart from the second pocket. The first pocket is sized and configured to receive a portion of the first battery pack, the second pocket is sized and configured to receive a portion of the second battery pack. The rigid hood may comprise a mask (not shown), a helmet 255, a helmet with shroud 260 and a helmet with a double shroud 265. The hood 150 including a material, the material comprises TYCHEM, plastic or polymer or any materials known in the art.

The hood assembly and/or hood 105 may comprise one or more openings or slits that is sized and configured to receive at least a portion of the head suspension frame, at least a portion of the base of the head suspension frame, a communications system to send and receive communications. The communications system may comprise 2-way headphones, blue tooth ear buds, etc., stethoscopes, and/or any combination thereof. In addition, the one or more openings may be sized and configured to exhaust the air, which a flow rate of the exhausted air is less than the flow rate of the blower to keep the positive pressure. The one or more openings or slits may comprise sealed component to prevent the ingress and/or egress of contaminant, the sealed component may be a Velcro flap or zipper, or a one-way or two-way valve (e.g., a pressure or air release valve). The one or more openings may be disposed within and/or adjacent to the top region of the hood and/or hood assembly. The one or more openings may be disposed within and/or adjacent to the back region of the hood assembly and/or hood. The one or more openings comprising a shape, the shape includes a circle, an oval, a regular polygon shape, an irregular polygon shape, or an elongated circle or elongated oval, and/or any combination thereof.

In one embodiment, the hood and/or hood assembly 105 comprises a first opening and a second opening. The first opening is sized and configured to receive a portion of the head suspension frame and/or a portion of the base of the head suspension frame. The first opening disposed in a back region of the hood 105. The second opening being disposed within a top region of the hood 105. The second opening being sized and configured to exhaust air, which a flow rate of the exhausted air is less than the flow rate of the blower to keep the positive pressure within the hood and/or hood assembly. The second opening may comprise a sealed component, the sealed component includes a one-way valve, a two-way valve, a port, and/or any combination thereof. The first and/or second openings comprising a shape, the shape includes a circle, an oval, a regular polygon shape, an irregular polygon shape, or an elongated circle or elongated oval, and/or any combination thereof.

FIGS. 16A-16F depicts various views of one embodiment of a hood plate 300.

The hood plate 300 improves structural rigidity near and/or adjacent to the crown region of a head of a wearer. The hood plate 300 also improves the containment and directionality of the air flow downward and towards the front region of the hood 105 to allow defogging to take place at the face shield. The hood plate 300 further provides a base for the head suspension frame 110 to mate and/or be coupled for enhanced or improved movement or swiveling independent of the entire hood assembly or hood 105. The hood plate 105 comprises a shape, the shape may be circular, oval and/or elliptical. The shape may match or substantially match the inner surface area of the hood. The hood plate 105 is disposed within the top region of the hood assembly and/or hood 105. The hood plate 105 further comprises a material, the material includes a plastic or metal. The hood plate 105 having a top surface and a bottom surface. The top surface facing towards an inner surface of the hood and/or hood assembly 105. The bottom surface facing towards a crown region of a head of a wearer.

FIGS. 16D-16F depict an isometric and side view of one embodiment of flow sensor 290. Accordingly, the at least one alarm may comprise an air flow sensor. The air flow sensor 290 may comprise tactile, visible and/or audible alarm. The air flow sensor 290 may comprise a thermal air flow sensor, a mass air flow sensor, a volume air flow sensor, a monometer, a moving air vane meter, a membrane sensor, a karman vortex sensor, a coldwire sensor, a telltales (a flexible assembly that comprises a string or fabric that moves and changes direction with the wind). The air flow sensor 290 may further comprises a telltale and/or a flexible assembly, the flexible assembly is disposed inside 315 the flexible hose 35 and/or disposed within an inner diameter 315 of the flexible hose 35. At least a portion of the flexible assembly extends beyond the first or second end of the flexible hose 35. At least a portion of the flexible assembly or telltale is movable from a first position to a second position, the first position being within the peripheral vision of a wearer to indicate low air flow or no air flow, and the second position being outside of the peripheral vision of the wearer to indicate sufficient air flow. The flexible assembly comprises a longitudinal member, the longitudinal member 288 comprising a string, a yarn, a fabric strips, recording tape, ribbons (e.g., nylon) and/or any combination thereof. The flexible assembly may further comprise a disc and/or a bead 292. The disc and/or bead 292 may be coupled to at least one end of the longitudinal member 288. Furthermore, the longitudinal member 288 including a first end and a second end, the second end being coupled within the air tube 35 and/or the inner diameter of the tube 35, at least a portion of the first end of the longitudinal member 288 extending beyond the second end of the flexible tube 35—the first end of the longitudinal member 288 being free. The disc or bead 292 being coupled to the first end of the longitudinal member 288.

FIG. 3F, FIGS. 5B-5D, 18A and 19A depicts different views of different embodiment of a toggle switch or on-off switch 97, 330. The toggle switch 97,330 is an electrical switch and/or a hinged switch that allows it to assume ON or OFF positions and making it possible to control the flow of electric current/signal from a power supply to the blower motor 30, 30a, 30b and any other electrical components within the PAPR units. The on-off switch or toggle switch 97, 330 may be disposed within the hood assembly and/or hood 105. The on-off switch or toggle switch 97, 330 may be disposed within and/or adjacent to the top region or crown region of the hood assembly 105. The on-off or toggle switch 97, 330 may be disposed within the hood assembly and/or hood 105 and within and/or adjacent to the back region of the hood and/or hood assembly 105. If the on-off switch is disposed within the hood assembly and/or hood, the on-off switch may be palpitated to by the user for location and activation. The on-off switch or toggle switch 97, 330 may be disposed adjacent to the PAPR unit. Alternatively, at least a portion of the on-off switch and/or toggle switch 97, 330 may be disposed within the filter base 25, 28, 330. The toggle switch 97, 330 may be disposed onto the housing 40, 40a, 40b of the PAPR unit.

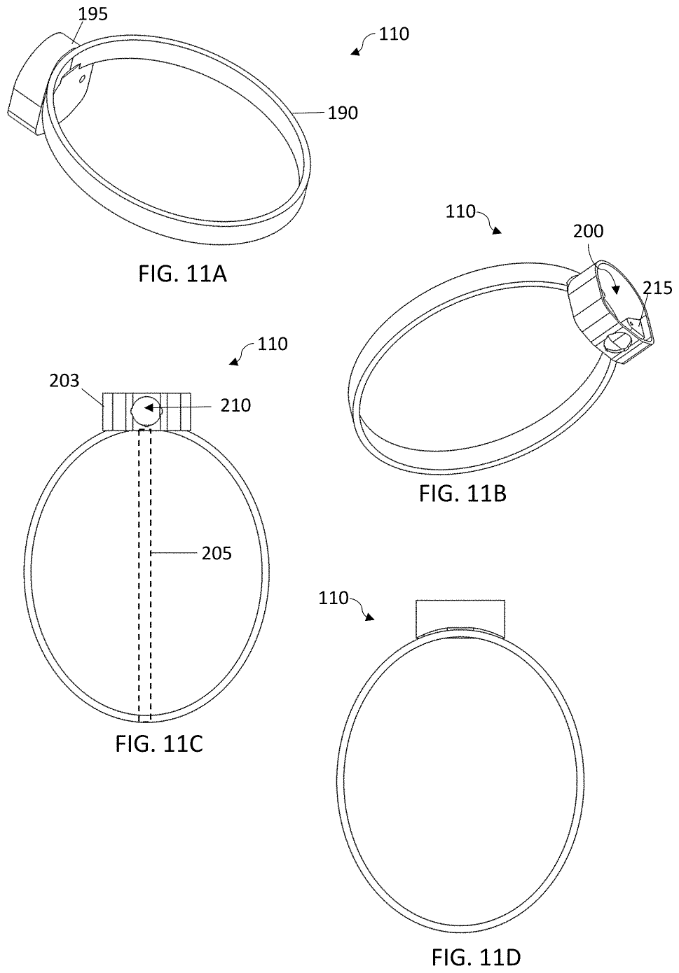

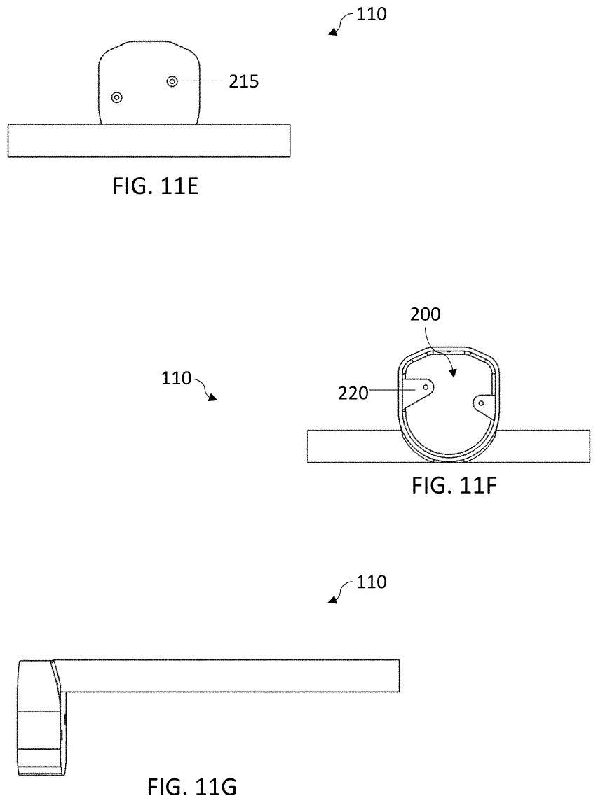

FIGS. 6, 11A-11G and 15A-15C depicts various views of different embodiments of a head suspension frame 110. The head suspension frame 110 may be adjustable around the circumference of a user's head and/or adjustable for the height on the user's head. The head suspension frame 110 may comprise a base 195 and a ring frame 190. The ring frame 190 may comprise a circumferential member, the circumferential member is adjustable around the circumference of the user's head. The head suspension frame 110 may further comprise a crown member 205, the crown member 205 positioned proximate to a user's crown region. The crown member 205 may be adjustable, the adjustability may comprise adjustment of the height from the crown of the user's head. The base 195, the ring frame 190 and/or the crown member 205 comprising a material, the material includes polymers, spring tempered metal, thermoplastic elastomers, thermoset elastomers, elastic, foam, and/or any combination thereof. At least a portion of the head suspension frame 110 is disposed within the hood assembly and/or the hood 105. The head suspension frame 110 may further comprise one or more comfort pads (not shown), the one or more comfort pads disposed on an inner surface of the head suspension frame 110. The head suspension frame may be positioned adjacent to the hood plate. Accordingly, the head suspension frame 110 may be positioned adjacent to a bottom surface of the hood plate.

The base 195 comprises a first surface and a second surface. The first surface comprises a recess 200. The second surface being coupled to a portion of the ring frame 190. The coupling may be fixed or rotatable or movable. The recess 200 is sized and configured to receive a portion of the blower motor 30 and/or the recess 200 is sized and configured to receive a second or first end of the blower motor 30. The base 195 may further comprises a sidewall 203, the sidewall 203 comprises an opening 210. The base 195 may further comprise securement tabs 220, the securement tabs 220 may extend outwardly within the recess 200 to help secure the blower motor 30 in place. The base 195 may further include rivets 215 on the second surface, the rivets 215 are spaced apart. The rivets 215 are spaced apart and offset or aligned (e.g., parallel).

The ring frame 195 may further comprise a channel 275. The channel 275 may be sized and configured to receive one or more battery pack assemblies 45,50. The ring frame 195 may further comprise a plurality of ring portions or circumferential members, at least one of the plurality of ring portions or circumferential members may comprise a channel 275. The channel 275 may be sized and configured to receive one or more battery pack assemblies 45,50. The channel 275 may be disposed throughout the entire length of the ring frame 195 and/or the channel 275 may be disposed only on a portion of the length of the ring frame 195.

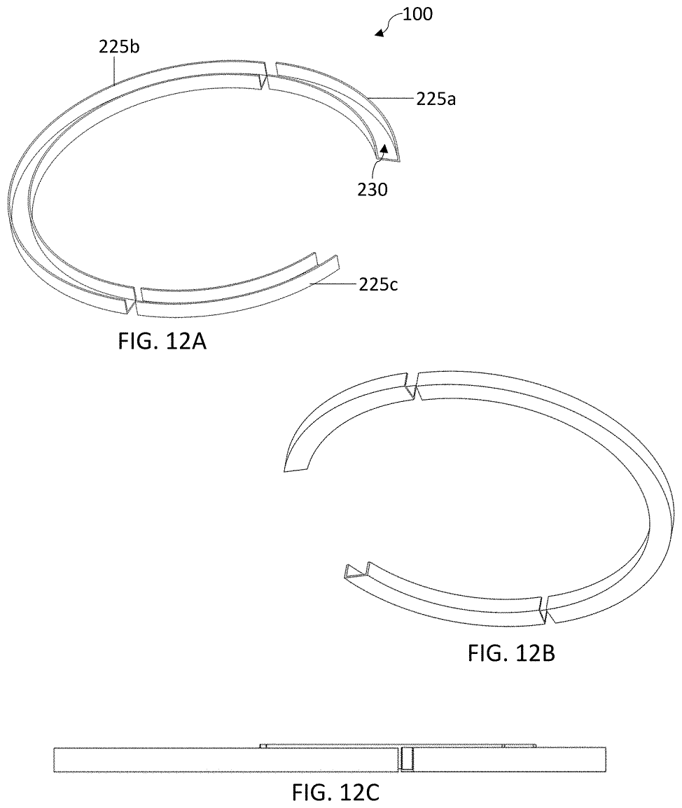

FIGS. 12A-12C depicts various views of one embodiment of a collar 100. The collar 100 may be disposed within the hood assembly and/or hood 105. The collar 100 may be disposed over the hood assembly and/or the hood 105. The collar 100 may be disposed on or over a neck or shoulders of a user. The collar 100 may be disposed adjacent or proximate to a neck or shoulders of a user. The collar 100 may be adjustable around the neck and/or shoulders of a user. The adjustability may allow the collar 100 to move from an untightened position to a tightened position. The tightened position may comprise and/or create a sealed barrier and/or a substantially sealed barrier to the user's neck. The sealed barrier includes allowing at least one surface of the collar 100 that contacts or mates with a portion of the user's neck region to prevent unfiltered air, particulates, blood or blood components, pathogens from passing (egress and ingress) between the barrier The substantial barrier includes allowing at least one surface of the collar that contacts or mates with a portion of the user's neck region to substantially deflect unfiltered air, particulates, blood or blood components, pathogens from passing through the barrier. The collar 100 may comprise one or more elongated members 225a, 225b, 225c, the one or more elongated members 225a, 225b, 225c may comprise a channel 230. The channel 230 may be sized and configured to receive a battery pack 45,50. Alternatively, the battery pack 45,50 may be disposed in portion of the collar 100. The collar 100 comprising a material, the material includes polymers, spring tempered metal, thermoplastic elastomers, thermoset elastomers, elastic, foam, and/or any combination thereof.

In various embodiments, the PAPR kit and/or PAPR unit may comprise an analog circuit (not shown). The analog circuit may comprise resistors and shunt resistors to eliminate the ability to require programmable software or an ASICS (programmable circuit). The resistors may be modified for different air flow (using voltage) warning levels, which triggers the at least one light to get dimmer for a further warning to user, then the at least one light will turn off for imminent air flow warning. The analog circuit will also be used to lower the air flow rate when the batteries near the end of their discharge cycle. The analog circuit may be disposed in a portion of the housing, in a portion of the collar, in a portion of the PAPR unit, in a portion of the hood assembly, within the hood assembly and adjacent to the top region, and/or in a portion of the head suspension frame. Alternatively, the circuit may comprise an ASICS (programmable circuit).

In various embodiments, the PAPR kit may comprise an alarm and/or at least one alarm. The at least one alarm may comprise a tactile, visible and/or audible alarm to notify user of low air flow and/or low battery voltage. The visible alarm may comprise at least one light (see FIGS. 5B-5E) or a telltale air flow sensor 290. The alarm may be disposed within the PAPR kit, the PAPR unit and/or the hood assembly. The wires for the alarm and/or the at least one light connecting wires may be disposed within the flexible hose, and should not impede air flow. The alarm may be disposed within the front region of the helmet. The alarm may be disposed within the front region of the helmet and within peripheral vision of the user. The alarm may be disposed within the hood assembly adjacent to the front region of the helmet. The alarm may be disposed within the housing of the PAPR unit. In another embodiment, the tactile alarm may comprise a vibratory mechanism.

In another embodiment, the PAPR kit may comprise a first alarm and a second alarm. The first alarm is the same or different that the second alarm. The first alarm and/or the second alarm comprises at least one of a tactile, visual and/or audible alarm. The first alarm may comprise a visual alarm, the second alarm may comprise a visual alarm. The first alarm may comprise at least one light, the at least one light includes an LED light. The second alarm may comprise an air flow sensor, the air flow sensor comprises a flexible assembly. The first alarm and/or the second alarm may be disposed within the front region of the hood and/or the hood assembly. Alternatively, the PAPR kit may comprise at least one alarm and an air flow sensor.

In various embodiments, the PAPR kit, the PAPR unit, the hood assembly and/or the hood, may comprise a pathogen inactivation coating. The pathogen inactivation coating may comprise methylene-blue (MB) impregnated dye or other coating (which may include coatings of nano-sized MB particulate coatings), which desirably photoactivates to provide a virus resistant surface in daylight (better faster activation) and in indoor lighting (lesser, slower activation). Methylene-blue can be applied and/or cured on various components, such as by spraying on or vapor deposition in a cabinet to produce nanoparticles, baking on with hot air flow or oven, etc. In various embodiments, a certain proportion of MB and binder agent or solvent can be utilized as a coating or paint. The pathogen activated coating may be activated by light (e.g., any types of bulbs or UV light up to a maximum absorption of 670 nm) allowing a limited useful life for protection of the user to a limited time, known as a degradation life cycle. The limited time may comprise a useful time of 8 to 16 hours, or 8 to 12 hours or 8 to 10 hours. The pathogen activation coating comprises anticoagulants, preservatives, germicidal agents, sterilants, antiseptics, clot activators, separator gels.

In various embodiments, MB can be useful to kill, damage and/or inactivate some or all of viruses and/or other microorganism that contact the various system components. An article entitled “Methylene blue-mediated photodynamic inactivation as a novel disinfectant of enterovirus 71” by Wong et al, J. Antimicrobe Chemotherapy (2010); 65: 2176-2182 (the disclosure of which is incorporated herein by reference in its entirety) described the use of MB in the presence of light as an inexpensive and safe photosensitizer for photodynamic inactivation of enterovirus 71 (EV71) in the environment, which Applicant believes may be useful in inactivating other viruses, such as COVID-19, SARS, MERS and/or other pathogens from the various components of the disclosed invention. Specifically, the Wong et al. paper looked at a total kill of a virus at 10 min and 200 J/cm\hat{ }2 light density. Outdoor light is 1200 J/cm\hat{ }2 and hospital lighting could be 200 J/cm\hat{ }2, 6500K color temperature light bulbs. With a wavelength of 632 nm which is also typical for indoor lighting. In some embodiments, a total Virus kill level at some energy/time level is disclosed, while in other embodiments a certain percentage of kill/virus resistance based on the light intensity/time may be useful to reduce virus load, such as outdoor usage where the virus resistance MB coated fabric may provide a 30% improvement in virus resistance/killing, while in indoor usage, because of the lower light intensity, the benefit may only be 5% to 10% (i.e., which could also be based on time). If the system is exposed to more time in the light, the percentage of resistance/killing would desirably increase.

Photodynamic treatment (PDT) has been previously applied to destroy cancer cells and a variety of microorganisms including viruses and bacteria by combining light and photosensitizing agents in the presence of oxygen. Methylene blue, a thiazine compound, has been used for decades for cell staining, delineating tumor margins during operations and as an antidote for nitrate poisoning and methaemoglobinaemia. Methylene blue has also been used as a photosensitizer in PDT. Methylene blue absorbs visible light between 620 and 670 nm. After photoactivation, methylene blue generates singlet oxygen and free oxygen radicals, which inactivate bacteria and viruses by damaging and/or cross-linking the nucleic acid and proteins. Methylene blue-mediated PDT (MB-PDT) has been applied in the sterilization of blood products because of several advantages including its high safety profile, low cost, minimal leftovers and low reduction of coagulation factors. It is proposed to utilize MB-PDT as an environment disinfectant on the various solid surfaces of the disclosed powered air filtering devices.

In various alternative embodiments, MB or similar compounds may desirably be incorporated into various other surfaces and/or components of the system, which may include system packaging such as a cardboard box and/or plastic wrapping surrounding the device. If desired, the packaging (i.e., cardboard and/or plastic bags) could include a MB coating or dye which desirably inactivates various viruses when exposed to light, such as during transport and/or shipment of the system by common carriers (i.e., FedEx, Amazon, United Parcel Service, US Postal Service, etc.) during a pandemic, where the package might be exposed and/or contaminated during such shipment. Once the package arrives at a desired destination, it may be intentionally left exposed to light (i.e., natural and/or artificial light) which induces a sterilization effect on the package and prevents accidental expose to live viruses by the recipient.

In some embodiments, the PAPR kit and/or PAPR unit and/or various components thereof may be packaged in airtight and/or opaque foil or other packaging (i.e., a “no light access” material or foil pouch), which will desirably prevent damaging moisture from entering the package and/or prevent light transmission which could inadvertently activate the pathogen inactivation coating. In at least one exemplary embodiment, a PAPR unit or various components thereof might be packaged in an opaque outer covering (i.e., a foil pouch or similar item) and further contained within an inner clear plastic pouch, where the PAPR unit can be removed from the foil pouch (yet remain within the clear pouch) and exposed to sunlight or artificial light, which might additionally sterilize the unit and/or greatly extend the useful sterile life of the device for an extended period of time (i.e., potentially for periods of 5, 10, 15 or 20 years of storage or greater).

In one embodiment, the pathogen inactivation coating comprises an application method onto a substrate for effective use. The pathogen inactivation coating application comprises the steps of: applying the pathogen inactivation coating in an aqueous state onto the substrate to allow penetration of coating into a plurality of pores of the substrate; measuring the viral penetration resistance or a resistance of substrate for penetration of pathogens through use of standard test method. The steps may further comprise confirming whether the pathogen inactivation coating coated the pores of a substrate. The steps may further comprise curing the pathogen inactivation coating to deactivate the coating effectiveness or performance and confirming whether the pathogen inactivation coating coated and/or penetrated the pores of a substrate. The pathogen inactivation coating may comprise phenothiazine dye and/or any of its derivatives. The pathogen inactivation coating may comprise any of the other coatings disclosed herein. The standard test method may comprise ASTM 1671. The substrate may comprise a surgical gown, a hood or hood assembly, a PAPR unit and/or a PAPR kit.

In another embodiment, the pathogen inactivation coating comprises a method of protection or method of performance. The method of protection for a pathogen inactivation coating includes the steps of: applying the pathogen inactivation coating in an aqueous state onto the substrate to allow penetration of coating into a plurality of pores of the substrate; curing the pathogen inactivation coating to decrease effectiveness of pathogen inactivation of the pathogen inactivation coating; absorbing an aqueous droplet comprising a pathogen onto a portion of a surface of the substrate and/or a localized region of the substrate; photo activating the pathogen inactivation coating effectiveness or performance by re-wetting and/or rehydrating the cured pathogen inactivation coating with the aqueous droplet comprising a pathogen by absorbing a light; and decreasing pathogen load or pathogen potential to suppress and/or lower pathogen infectiousness. The photo activation may include inhibitory activity against the interaction between the SARS-CoV-2 spike protein and its cognate receptor ACE2, which is the first critical step initiating the viral attachment and entry of the Coronavirus. The light may comprise UV-A, UV-B, UV-C and/or LED lights.

In another embodiment, the pathogen inactivation coating comprises a method of protection. The method of protection for a pathogen inactivation coating includes the steps of: applying the pathogen inactivation coating in an aqueous state onto the substrate to allow penetration of coating into a plurality of pores of the substrate; curing the pathogen inactivation coating to decrease effectiveness of pathogen inactivation of the pathogen inactivation coating; absorbing an aqueous droplet comprising a pathogen onto a portion of a surface of the substrate and/or a localized region of the substrate; activating the pathogen inactivation coating effectiveness or performance by re-wetting and/or rehydrating the cured pathogen inactivation coating with the aqueous droplet comprising a pathogen; and decreasing pathogen load or pathogen potential to suppress and/or lower pathogen infectiousness. The activation may include inhibitory activity against the interaction between the SARS-CoV-2 spike protein and its cognate receptor ACE2, which is the first critical step initiating the viral attachment and entry of the Coronavirus. The light may comprise UV-A, UV-B, UV-C and/or LED lights.

The light or light wavelength may comprise UV-A, UV-B, UV-C and LED light. The UV-A wavelength comprises a wavelength range of 315 to 400 nanometers. The UV-B wavelength comprises a wavelength range of 280 to 315 nanometers. The UV-C wavelength comprises a wavelength range of 100 to 280 nanometers. In another embodiment, the wavelength comprises a range of 100 to 450 nanometers; the wavelength comprises 100 to 300 nanometers; the wavelength comprises 200 to 300 nanometers and specifically, the range may comprise 222 to 280 nanometers, the range may comprise 250 to 280 nanometers, the range may comprise 315 to 400 nanometers. Alternatively, the wavelength comprises 264 nanometers. Moreover, the pathogen inactivation coating may comprise phenothiazine dye and/or any of its derivatives. The pathogen inactivation coating may comprise any of the other coatings disclosed herein. The substrate may comprise a surgical gown, a hood or hood assembly, a PAPR unit and/or a PAPR kit.

In various alternative embodiments, components of the disclosed powered air-purifying respirator may be useful in other applications, such as applications where an enclosed and/or semi-enclosed space might benefit from negative ventilation (i.e., containing an infected patient or specimens) or from positive ventilation (i.e., containing uninfected patients or sterilized items). Such systems could create a desired level of negative ventilation in a room or vehicle where the pump is positioned within the room and the air tube extends outside a window or the door (i.e., with some sealant such as duct tape or other approved sealants covering various air gaps or such, as desired). The filtration unit will desirably capture and/or inhibit virus-containing particles as the air is pumped out of the room, creating a desired negative pressure airflow in the room (for the duration of battery power or other power supply, including mobile and/or fixed power supplies). Such a system could be very useful in emergency field hospitals and/or hospital buildings, as well as for ambulances and/or patient transport buses or the like. Alternatively, a reversed arrangement of the pump and air tubing could be utilized to maintain positive pressure within a room or vehicle, desirably protecting the occupants from virus-containing particles as the filtered air is pumped into the room, and creating a desired positive pressure airflow outward that prevents virus-containing particles and/or other pyrogens/pollutants (i.e., including dust, smoke and/or smog) from entering air gaps into the room or vehicle. In vehicle or other application, such system components may be powered by existing DC power systems (i.e., 12-volt systems or stepped-down AC/DC voltage systems), if desired, with battery back-up provided where needed.

In various embodiments, the PAPR kit and/or PAPR unit can be single use and/or disposable in total. Alternatively, the PAPR kit and/or PAPR unit may be reusable and/or cleanable/sterilizable, if desired. Single Use/Disposable components can lessen the opportunity for further disease transmission and/or the need for decontamination effort.

### Different Embodiments

In one embodiment, the PAPR kit comprising: a PAPR unit, the PAPR unit including a filter media, a blower and a battery pack; a hood assembly, the hood assembly comprising a hood, the hood including a pathogen activation coating, the pathogen activation coating comprising a photochemical effect, the photochemical effect being activated by a light and providing a limited degradation time cycle; and a flexible hose, the flexible hose comprising a first end and a second end, the first end being coupled to a portion of the PAPR unit, the second end being coupled to a portion of the hood assembly.

The filter media comprising a P95 filter, a P100 filter, an HE filter, a HEPA filter, and/or any combination thereof. The battery pack comprises rechargeable batteries. The battery pack being removably connected from the PAPR unit, the hood comprising a flexible hood or a rigid hood. The flexible hood comprising a head cover, a hood with a shroud, a hood with a double shroud, a sealed seam hood with a double shroud, and/or any combination thereof. The hood assembly further comprises a head suspension frame. The PAPR kit further comprises at least one light. The limited degradation time cycle comprises a range of 8 hours to 16 hours. The PAPR kit further comprises a stand-alone recharging kit. The PAPR kit further comprises an integrated solar recharging kit, the integrated solar recharging kit being disposed on a portion of the hood assembly.

In another embodiment, the PAPR kit comprises: a PAPR unit, the PAPR unit including a housing, a filter media, a blower and a battery pack, at least a portion of the battery pack being disposed within the housing; a hood assembly, the hood assembly comprising a hood, a hose receiver, at least one light, the at least one light being disposed in a front region of the hood; and a flexible hose, the flexible hose comprising a first end and a second end, the first end being coupled to a portion of the PAPR unit, the second end being coupled to the hose receiver.

The filter media comprising a P95 filter, a P100 filter, an HE filter, a HEPA filter, and/or any combination thereof. The battery pack comprises rechargeable batteries. The battery pack being removably connected from the housing. The hood comprising a flexible hood or a rigid hood. The flexible hood comprising a head cover, a hood with a shroud, a hood with a double shroud, a sealed seam hood with a double shroud, and/or any combination thereof. The hood assembly further comprises a head suspension frame. The PAPR kit further comprises a stand-alone AC recharging kit or a stand-alone solar charging kit. The hood comprises a pathogen activation coating. The pathogen activation coating is a photochemical coating, the photochemical coating comprising a photochemical effect, the photochemical effect being activated by a light and providing a limited degradation time cycle. The limited degradation time cycle comprises a range of 8 hours to 16 hours.

In another embodiment, the PAPR kit comprises: a PAPR unit, the PAPR unit including a filter media and a blower; a hood assembly, the hood assembly comprising a hood, and at least one light, the at least one light being disposed in a front region of the hood; a head suspension frame, at least a portion of the head suspension frame being disposed within the hood, the head suspension frame comprising a ring frame and a base, the base having a first opening and a second opening, the frame being adjustable around the circumference of a user's head, at least a portion of the PAPR unit being coupled to the second opening; a flexible hose, the flexible hose comprising a first end and a second end, the first end being coupled to the first opening; and a battery pack, the battery pack being disposed within at least a portion of the head suspension frame.

The filter media comprising a P95 filter, a P100 filter, an HE filter, a HEPA filter, and/or any combination thereof. The battery pack comprises rechargeable batteries. The battery pack being removably connected from the head suspension frame. The hood comprising a flexible hood or a rigid hood. The flexible hood comprising a head cover, a hood with a shroud, a hood with a double shroud, a sealed seam hood with a double shroud, and/or any combination thereof. The PAPR kit further comprises a stand-alone AC recharging kit or a stand-alone solar charging kit. The hood comprises a pathogen activation coating. The pathogen activation coating is a photochemical coating, the photochemical coating comprising a photochemical effect, the photochemical effect being activated by a light and providing a limited degradation time cycle. The limited degradation time cycle comprises a range of 8 hours to 16 hours. The PAPR kit further comprises an integrated solar recharging kit, the integrated solar recharging kit being disposed on a portion of the hood assembly.

In another embodiment, the PAPR kit comprising: a PAPR unit, the PAPR unit including a filter media and a blower; a hood assembly, the hood assembly comprising a hood, and at least one light, the at least one light being disposed in a front region of the hood; a head suspension frame, at least a portion of the head suspension frame being disposed within the hood, the head suspension frame comprising a ring frame and a base, the base having a first opening and a second opening, the frame being adjustable around the circumference of a user's head, at least a portion of the PAPR unit being coupled to the second opening; a flexible hose, the flexible hose comprising a first end and a second end, the first end being coupled to the first opening; a collar, the collar comprising one or more members, at least a portion of the one or more members having a channel; and a battery pack, the battery pack being disposed into portion of the channel of the collar.

The collar is adjustable, the adjustability allows the one or more members to move from a tightened position to an untightened position. The collar is flexible. The collar is disposed within the hood. The collar is disposed over the hood. The filter media comprising a P95 filter, a P100 filter, an HE filter, a HEPA filter, and/or any combination thereof. The battery pack comprises rechargeable batteries. The battery pack being removably connected from the head suspension frame. The hood comprising a flexible hood or a rigid hood. The flexible hood comprising a head cover, a hood with a shroud, a hood with a double shroud, a sealed seam hood with a double shroud, and/or any combination thereof. The PAPR kit further comprises a stand-alone AC recharging kit or a stand-alone solar charging kit. The hood comprises a pathogen activation coating. The pathogen activation coating is a photochemical coating, the photochemical coating comprising a photochemical effect, the photochemical effect being activated by a light and providing a limited degradation time cycle. The limited degradation time cycle comprises a range of 8 hours to 16 hours.

In another embodiment, the PAPR kit comprises: a PAPR unit, the PAPR unit including a filter media and a blower; a hood assembly, the hood assembly comprising a hood, and at least one light, the at least one light being disposed in a front region of the hood; a head suspension frame, at least a portion of the head suspension frame being disposed within the hood, the head suspension frame comprising a ring frame and a base, the base having a first opening and a second opening, the ring frame being adjustable around the circumference of a user's head, at least a portion of the PAPR unit being coupled to the second opening of the base; and a battery pack, the battery pack being disposed within at least a portion of the head suspension frame.

In another embodiment, the PAPR kit comprises: a PAPR unit, the PAPR unit including a filter media and a blower; a hood assembly, the hood assembly comprising a hood, and at least one light, the at least one light being disposed in a front region of the hood; a head suspension frame, at least a portion of the head suspension frame being disposed within the hood, the head suspension frame comprising a ring frame and a base, the base having a first opening and a second opening, the ring frame being adjustable around the circumference of a user's head, at least a portion of the PAPR unit being coupled to the second opening, the first opening being free to exhaust air; a collar, the collar comprising one or more members, at least a portion of the one or more members having a channel; and a battery pack, the battery pack being disposed into a portion of the channel of the collar.

In another embodiment, the surgical PAPR kit comprises: a PAPR unit, the PAPR unit including a filter media and a blower, the blower including a blower air flow rate; a hood assembly, the hood assembly comprising a hood, and at least one light, the at least one light being disposed in a front region of the hood, the hood including at least one opening, the at least one opening being disposed within the top region of the hood, the at least one opening being sized and configured to have an exhaust flow rate less than the blower air flow rate to maintain positive pressure within the hood assembly; a head suspension frame, at least a portion of the head suspension frame being disposed within the hood, the head suspension frame comprising a ring frame and a base, the base having a first opening and a second opening, the frame being adjustable around the circumference of a user's head, at least a portion of the PAPR unit being coupled to the second opening of the base; and a flexible hose, the flexible hose comprising a first end and a second end, the first end being coupled to the first opening of the base, the second end being free and positioned adjacent to the front region of the hood; a collar, the collar being adjustable around a user's neck to create a sealed barrier or a substantially sealed barrier; and a battery pack, the battery pack being disposed into at least a portion of the collar or at least a portion of the head suspension frame.

In another embodiment, the surgical PAPR kit comprises: a PAPR unit, the PAPR unit including a filter media and a blower, the blower including a blower air flow rate; a hood assembly, the hood assembly comprising a hood, and at least one light, the at least one light being disposed in a front region of the hood, the hood including at least one opening, the at least one opening being disposed within the top region of the hood, the at least one opening being sized and configured to have an exhaust flow rate less than the blower air flow rate to maintain positive pressure within the hood assembly; a head suspension frame, at least a portion of the head suspension frame being disposed within the hood, the head suspension frame comprising a ring frame and a base, the base having a first opening and a second opening, the frame being adjustable around the circumference of a user's head, at least a portion of the PAPR unit being coupled to the second opening of the base, the first opening being free to provide the air flow rate of the blower; a collar, the collar being adjustable around a user's neck to create a sealed barrier or a substantially sealed barrier; and a battery pack, the battery pack being disposed into a portion of the collar or a portion of the head suspension frame.

In another embodiment, the PAPR kit comprises: a PAPR unit, the PAPR unit including a first filter media, a second filter media and a blower, the first filter media having a first axis and the second filter media having a second axis; a hood, the hood comprising a shroud; a head suspension frame, the head suspension frame being adjustable around the circumference of a user's head, the head suspension frame disposed within a top region of the hood; a flexible hose, the flexible hose comprising a first end and a second end, the second end of the flexible hose being free and disposed near the front region of the hood; an acoustic tube, the acoustic tube comprising a first end and a second end, the first end of the acoustic tube coupled to a portion of the blower, the second end of the acoustic tube coupled to the first end of the flexible hose; at least first alarm and a second alarm; and a plurality of battery packs.

In another embodiment, the PAPR kit comprises: a PAPR unit, the PAPR unit including a first filter media, a second filter media and a blower, the first filter media having a first axis and the second filter media having a second axis, the first axis of the first filter media is spaced apart and parallel to the second axis of the second filter media; a hood, the hood comprising a shroud; a head suspension frame, the head suspension frame being adjustable around the circumference of a user's head, the head suspension frame disposed within a top region of the hood; and a flexible hose, the flexible hose comprising a first end and a second end, the second end of the flexible hose being free and disposed near the front region of the hood.

In another embodiment, the PAPR kit comprises: a PAPR unit, the PAPR unit including a first filter media, a second filter media and a blower, the first filter media having a first axis and the second filter media having a second axis, the first axis of the first filter media is spaced apart and parallel to the second axis of the second filter media; a hood, the hood comprising a shroud; a head suspension frame, the head suspension frame being adjustable around the circumference of a user's head, the head suspension frame disposed within a top region of the hood; an acoustic tube, the acoustic tube having a first end and a second end, the first end being coupled to at least a portion of the blower; and a flexible hose, the flexible hose comprising a first end and a second end, the second end of the flexible hose being free and disposed near the front region of the hood, the first end of the flexible hose being coupled to the second end of the acoustic tube/

The first axis of the first filter media is parallel to the second axis of the second filter media. The first axis of the first filter media is colinear to the second axis of the second filter media. The PAPR kit further comprises a hood plate, the hood plate positioned adjacent to the head suspension frame. The PAPR kit further comprises an acoustic tube, the acoustic tube comprising a first end and a second end, the first end of the acoustic tube is coupled to a portion of the blower, and the second end of the acoustic tube is coupled to a first end of the flexible tube. The acoustic tube is a closed cell foam. The PAPR kit further comprising a first alarm and a second alarm. The first alarm comprises at least one light, the at least one light disposed within the front region of the hood. The at least one light comprises an LED light. The second alarm comprises an air flow sensor. The air flow sensor comprises a flexible assembly, the flexible assembly is disposed within the flexible hose, at least a portion of the flexible assembly extends beyond the second end of the flexible hose. The at least a portion of the flexible assembly is movable from a first position to a second position, the first position being within the peripheral vision of a wearer to indicate low air flow or no air flow, and the second position being outside of the peripheral vision of the wearer to indicate sufficient air flow.

The PAPR unit is disposed within a back region of the hood. The PAPR unit is disposed within a back region of the hood and adjacent to the neck and shoulder region. The PAPR unit is disposed within a back region of the shroud of the hood. The PAPR unit is disposed adjacent and/or within the waist region. The first filter media and the second filter media comprising a P95 filter, a P100 filter, an HE filter, a HEPA filter, and/or any combination thereof. The first filter media comprises the same or different filter media compared to second filter media.

I/We claim:

1. A PAPR kit comprising:
a hood, the hood comprising a shroud;
a PAPR unit, the PAPR unit including a blower and a filter assembly, the filter assembly comprising a filter base, a first filter media and a second filter media, the filter base comprising a top end and a bottom end, the top end of the filter base including a recess, the first and second filter media disposed within the recess, at least a portion of the blower coupled to a portion of the bottom end of the filter base;
a head suspension frame, the head suspension frame being adjustable around the circumference of a user's head, the head suspension frame disposed within a top region of the hood;
a flexible hose, the flexible hose comprising a first end and a second end, the second end of the flexible hose being free and disposed near the front region of the hood, the first end coupled to a portion of the blower of the PAPR unit; and
at least one battery pack.

2. The PAPR kit of claim 1, wherein the PAPR kit is sterilizable and disposable.

3. The PAPR kit of claim 1, wherein the first filter media and the second filter media comprise a same filter media.

4. The PAPR kit of claim 2, wherein the same filter media comprises a PM 2.5 filter, a P95 filter, an N95 filter, a KN95 filter, a P100 filter, a HEPA filter, an HE filter, and/or any combination thereof.

5. The PAPR kit of claim 1, wherein the PAPR kit further comprises an acoustic tube to dampen noise and vibration of the blower, at least a portion of the acoustic tube coupled to the flexible hose and positioned adjacent to the first end of the flexible hose.

6. The PAPR kit of claim 1, wherein the PAPR kit further comprises at least one light, the at least one light disposed within a front region of the hood adjacent to a wearer's peripheral vision.

7. The PAPR kit of claim 1, wherein the PAPR kit further comprises an air flow sensor, the air flow sensor includes a telltale assembly, the telltale assembly includes a first end and a second end, the second end of the telltale is coupled to a second end of the flexible hose, the first end of the telltale extends beyond the second end of the flexible hose adjacent to a user's peripheral vision, the at least a portion of the telltale assembly is movable from a first position to a second position, the first position being within the peripheral vision of the user to indicate low air flow or no air flow, and the second position being outside of the peripheral vision of the user to indicate sufficient air flow.

8. The PAPR kit of claim 1, wherein at least a portion of the blower of the PAPR unit is disposed within the hood and downstream from the filter assembly of the PAPR unit.

9. The PAPR kit of claim 1, wherein at least a portion of the PAPR kit further comprises a pathogen inactivation coating, the pathogen inactivation coating includes a photoactivation coating, a germicidal coating, or a disinfectant coating.

10. The PAPR kit of claim 9, wherein the photoactivation coating comprises phenothiazine dye or a phenothiazine derivative.

11. The PAPR kit of claim 10, wherein the phenothiazine derivative comprises methylene blue.

12. A PAPR kit comprising:
a PAPR unit, the PAPR unit including a filter assembly and at least one blower, the filter assembly comprising a filter base and at least one filter media, the filter base including a top surface and a bottom surface, the top surface including a recess, the at least one filter media disposed within the recess, at least a portion of the blower coupled to a portion of the bottom surface of the filter base;
a hood, the hood comprising a shroud, the hood comprising a front region, a back region, a top region, a bottom region, an inner surface and an external surface;
a head suspension frame, the head suspension frame being adjustable around the circumference of a user's head, the head suspension frame disposed within the top region of the hood;
a flexible hose, the flexible hose comprising a first end and a second end, the second end being coupled to the PAPR unit and the first end coupled to the hood; and
at least one battery pack.

13. The PAPR kit of claim 12, wherein the PAPR kit further comprises an acoustic tube to dampen noise and vibration of the blower, at least a portion of the acoustic tube disposed between the first end and a second end of the flexible hose.

14. The PAPR kit of claim 12, wherein the PAPR kit further comprises at least one light, the at least one light disposed within a front region of the hood adjacent to a user's peripheral vision.

15. The PAPR kit of claim 12, wherein the at least one light comprises a UV light, the UV light includes a UV-A, a UV-B, a UV-C and/or any combination thereof.

16. The PAPR kit of claim 12, wherein the hood comprising a pathogen coating, the pathogen coating includes a phenothiazine dye or a phenothiazine derivative.

17. The PAPR kit of claim 12, wherein the PAPR unit disposed in a back region of the hood.

18. The PAPR kit of claim 16, wherein a portion of the PAPR unit is disposed on an inner surface of the back region of the hood.

19. The PAPR kit of claim 16, wherein a first portion of the PAPR unit is disposed on an inner surface of the back region of the hood and a second portion disposed on an external surface.

20. The PAPR kit of claim 12, wherein the at least one battery pack is disposed in the front region of the hood.
